(19) United States
(12) Patent Application Publication
(10) Pub. No.: US 2005/0136549 A1
(43) Pub. Date: Jun. 23, 2005

(54) Method and system for automatically determining diagnostic saliency of digital images

(75) Inventors: Abhijeet S. Gholap, San Jose, CA (US); Gauri A. Gholap, San Jose, CA (US); C. V. K. Rao, San Jose, CA (US); Sanford H. Barsky, Los Angeles, CA (US); Prithviraj Jadhav, San Jose, CA (US); Jayant Abhyankar, San Jose, CA (US); Madhura Vipra, Prune (IN)

Correspondence Address: LESAVICH HIGH-TECH LAW GROUP, P.C., SUITE 325, 39 S. LASALLE STREET, CHICAGO, IL, 60603, US

(73) Assignee: Bioimagene, Inc., San Jose, CA (US)

(21) Appl. No.: 10/966,071

(22) Filed: Oct. 15, 2004

Related U.S. Application Data
(60) Provisional application No. 60/515,582, filed on Oct. 30, 2003.
(60) Provisional application No. 60/530,174, filed on Dec. 15, 2003.

(51) Int. Cl. C12Q 1/00; G01N 33/566

(52) U.S. Cl. 436/501

(57) ABSTRACT

A method and system for automatically determining diagnostic saliency of digital images for medical and/or pathological purposes. Luminance parameters (e.g. intensity, etc.) from a digital image of a biological sample (e.g., tissue cells) to which a chemical compound (e.g., a marker dye) has been applied are automatically analyzed and automatically corrected if necessary. Morphological parameters (e.g., cell membrane, cell nucleus, mitotic cells, etc.) from individual components within the biological sample are automatically analyzed on the digital image. A medical conclusion (e.g., a medical diagnosis or prognosis) is automatically determined from the analyzed luminance and morphological parameters.

### CROSS REFERENCES TO RELATED APPLICATIONS

This application claims priority from U.S. patent application Ser. No. 10/938,314, filed Sep. 10, 2004, which claims priority U.S. Provisional Patent Application No. 60/501,142, filed Sep. 10, 2003, and U.S. Provisional Patent Application No. 60/515,582 filed Oct. 30, 2003, and this application and also claims priority from U.S. Provisional Patent Application No. 60/515,582 filed Oct. 30, 2003, and U.S. Provisional Patent Application No. 60/530,714, filed Dec. 18, 2003, the contents of all of which are incorporated by reference.

### COPYRIGHT NOTICE

Pursuant to 37 C.F.R. 1.71(e), applicants note that a portion of this disclosure contains material that is subject to and for which is claimed copyright protection, such as, but not limited to, digital photographs, screen shots, user interfaces, or any other aspects of this submission for which copyright protection is or may be available in any jurisdiction. The copyright owner has no objection to the facsimile reproduction by anyone of the patent document or patent disclosure, as it appears in the U.S. Patent Office patent file or records. All other rights are reserved, and all other reproduction, distribution, creation of derivative works based on the contents of the application or any part thereof are prohibited by applicable copyright law.

### FIELD OF THE INVENTION

This invention relates to digital image processing. More specifically, it relates to a method and system for automatically determining diagnostic saliency of digital images.

### BACKGROUND OF THE INVENTION

As is known in the art, medical, life science and biotechnology experiments typically produce large amounts of digital information and digital images. Such experiments include study in disciplines such as genomics, proteomics, pharmacogenomics, molecular imaging, diagnostic medical imaging includes histopathology, cell-cycle analysis, genetics, magnetic resonance imaging (MRI), digital x-ray and computed tomography (CT). Converting large amounts raw data including raw data on digital images generated in these experiments into meaningful information that can be used by an analyst to formulate an opinion remains a challenge that hinders many investigators.

As is known in the art, genomics is the study of genomes, which includes genome mapping, gene sequencing and gene function. Gene expression microarrays are revolutionizing the biomedical sciences. A DNA microarray consists of an orderly arrangement of DNA fragments representing the genes of an organism. Each DNA fragment representing a gene is assigned a specific location on the array, usually a glass slide, and then microscopically spotted (<1 mm) to that location. Through the use of highly accurate robotic spotters, over 30,000 spots can be placed on one slide, allowing molecular biologists to analyze virtually every gene present in a genome. A complementary DNA (cDNA) array is a different technology using the same principle; the probes in this case are larger pieces of DNA that are complementary to the genes one is interested in studying. High-throughput analysis of micro-array data requires efficient frame work and tools for analysis, storage and archiving voluminous image data. For more information see “DNA Microarrays. History and overview” by E. M. Southern, Methods Molecular Biology Journal, 170: 1-15, 2001.

As is known in the art, proteomics is the study of the function of expressed proteins and analysis of complete complements of proteins. Proteomics includes the identification and quantification of proteins, the determination of their localization, modifications, interactions, activities, and, ultimately, their function. In the past proteomics is used for two-dimensional (2D) gel electrophoresis for protein separation and identification. Proteomics now refers to any procedure that characterizes large sets of proteins. Rapid growth of this field is driven by several factors—genomics and its revelation of more and more new proteins; powerful protein technologies, such as newly developed mass spectrometry approaches, global two-hybrid techniques, and spin-offs from DNA arrays. See for example, “From genomics to proteomics,” by M. Tyers and M. Mann in Nature Journal 2003, 13:422(6928):193-7. Large-scale data sets for protein-protein interactions, organelle composition, protein activity patterns and protein profiles in cancer patients are generated in the past few years. Rapid analysis of these data sets requires innovative information driven framework and tools to process, analyze, and interpret prodigious amounts of data.

Tissuemicroarrays (TMA) work on the similar principles of DNA microarrays where large number of tissue samples are placed on a single slide and analyzed for these expression of proteins. The image data generated in such cases is tremendous and require efficient software analysis tools. TMA analysis involves reporting protein to be detected by immunohistochemical (IHC), immunofluorescence, luminescence, absorbance, and reflection detection.

As is known in the art, pharmacogenomics is the field of investigation that aims to elucidate the inherited nature of inter-individual differences in drug disposition and effects, with the ultimate goal of providing a stronger scientific basis for selecting the optimal drug therapy and dosages for each patient. There is great heterogeneity in the way humans respond to medications, often requiring empirical strategies to find the appropriate drug therapy for each patient. There has been great progress in understanding the molecular basis of drug action and in elucidating genetic determinants of disease pathogenesis and drug response. These genetic insights should also lead to mechanism-based approaches to the discovery and development of new medications. See, for example, “Pharmacogenomics: Unlocking the Human Genome for Better Drug Therapy,” by Howard L. McLeod, William E. Evans in Annual Review of Pharmacology and Toxicology 2001, Vol. 41: 101-121. Collection, analysis and maintenance of inter-individual differences data sets requires efficient information driven framework and tools to process, analyze, and interpret prodigious amounts of data.

Microscopy and molecular imaging include the identification of changes in the cellular structures indicative of disease remains the key to the better understanding in medicinal science. Microscopy applications as applicable to microbiology (e.g., gram staining), Plant tissue culture, animal cell culture (e.g. phase contrast microscopy), molecular biology, immunology (e.g. ELISA), cell biology (e.g., immunofluorescence, chromosome analysis) Confocal microscopy: Time-Lapse and Live Cell Imaging, Series and Three-Dimensional (3D) Imaging. The advancers in confocal microscopy have unraveled many of the secrets occurring within the cell and the transcriptional and translational level changes can be detected using fluorescence markers. One advantage of the confocal approach results from the capability to image individual optical sections at high resolution in sequence through a specimen. Framework with tools for 3D analysis of thicker sections, differential color detection, fluorescence in situ hybridization (FISH) etc., is needed to expedite the progress in this area.

Near infrared (NIR) multiphoton microscopy is becoming a novel optical tool for fluorescence imaging with high spatial and temporal resolution, diagnostics, photochemistry and nanoprocessing within living cells and tissues. NIR lasers can be employed as the excitation source for multifluorophor multiphoton excitation and hence multicolour imaging. In combination with FISH, this novel approach can be used for multi-gene detection (multiphoton multicolour FISH). See, for example, “Multiphoton microscopy in life sciences” by Konig K. in Journal of Microscopy, 2000, Vol. 200 (Part 2):83-104.

In-vivo imaging: Animal models of cancer are inevitable in studies that are difficult or impossible to perform in people. Imaging of in-vivo markers permit observations of the biological processes underlying cancer growth and development. Functional imaging—the visualization of physiological, cellular, or molecular processes in living tissue—would allows to study metabolic events in real time, as they take place in living cells of the body.

Diagnostic medical imaging: Imaging technology has broadened the range of medical options in exploring untapped potential for cancer diagnosis. X-ray mammography already has had a lifesaving effect in detecting some early cancers. Computed tomography (CT) and ultrasound permit physicians to guide long, thin needles deep within the body to biopsy organs, often eliminating the need for an open surgical procedure. CT scan images can reveal whether a tumor has invaded vital tissue, grown around blood vessels, or spread to distant organs; important information that can help guide treatment choices. Three dimensional image reconstruction and visualization techniques require significant processing capabilities using smaller, faster, and more economical computing solutions.

In the field of Histopathology including oncology, the detection, identification, quantification and characterization of cells of interest, such as cancer cells, through testing of biological samples is an important aspect of experimentation. Typically, a tissue sample is prepared by staining the tissue with dyes to identify cells of interest.

Examination of biological tissues typically has been performed manually by either a lab technician or a pathologist or a life science and biotechnology researcher. In the manual method, a slide prepared with a biological sample is viewed at a low magnification under a microscope to visually locate candidate cells of interest. Those areas of the slide where cells of interest are located are then viewed at a higher magnification to confirm those objects as cells of interest, such as tumor or cancer cells.

Diagnostic methods in pathology carry out the detection, identification, quantification and characterization of cells of interest. For example, in oncology, detection of cancer cells can be done by various methods, such as contrast enhancement by different dyes or by using a specific probe such an monoclonal antibody that reacts with component of cells of interest or by probes that are specific for nucleic acids.

In the last few years, slides with stained biological samples are photographed to create digital images from the slides. Digital images are typically obtained using a microscope and capturing a digital image of a magnified biological sample.

The ability to detect, through imaging, the histopathological image data for the molecular and phenotypic changes associated with a tumor cell will enhance pathologists ability to detect and stage tumors, select appropriate treatments, monitor the effectiveness of a treatment, and determine prognosis.

Cancer is an especially pertinent target of micro-array technology due to the well-known fact that this disease causes, and may even be caused by, changes in gene expression. Micro-arrays are used for rapid identification of the genes that are turned on and the genes that are turned off in tumor development, resulting in a much better understanding of the disease. For example, if a gene that is turned on in that particular type of cancer is discovered, it may be targeted use in cancer therapy. Today, therapies that directly target malfunctioning genes are already in use and showing exceptional results. Micro-arrays are also used for studying gene interactions including the patterns of correlated loss and increase of gene expression. Gene interactions are studied during drug design and screening. Large number of gene interactions studied during a drug discovery requires efficient frame work and tools for analysis, storage and archiving voluminous image data.

A standard test used to measure protein expression is immunohistochemistry (IHC). Analyzing the tissue samples stained with IHC reagents has been the key development in the practice of pathology. Normal and diseased cells have certain physical characteristics that can be used to differentiate them from each other. These characteristics include complex patterns, rare events, and subtle variations in color and intensity.

Hematoxillin and Eosin (H/E) method of staining is used to study the morphology of tissue samples. Based on the differences and variations in the patterns from the normal tissue, the type of cancer is determined. Also the pathological grading or staging of cancer (Richardson and Bloom Method) is determined using the H/E staining. This pathological grading of cancer is not only important from diagnosis angle but has prognosis value attached to it

As is known in the medical arts, an over expression of proteins can be used to indicate the presence of certain medical diseases. For example, in approximately 20%-30% patients with breast cancer, tumor cells show an amplification and/or over expression of human epidermal receptor-2 (HER-2), a tyrosine kinase receptor. HER-2 is a human epidermal growth factor receptor, which is also known as c-erbB-2/neu. HER-2/neu (C-erbB2) is a proto-oncogene that localizes to chromosome 17q. Protein product of this gene is typically over-expressed in breast cancers. This over expression in majority of cases (e.g., 90%-95%) is a direct result of gene amplification. Over expression of HER-2/neu protein thus has prognostic significance for mammary carcinoma.

Clinical studies in patients with breast cancer over the last decade have convincingly demonstrated that amplification/over expression of HER-2/neu is associated with a poor medical prognosis. Approximately 20%-30% of invasive breast carcinomas are HER-2/neu amplified. It has also been shown to be increased in a variety of other human malignancies including that of kidney, bladder and ovary. Gene amplification of HER-2/neu is associated with aggressive cell behavior and poor prognosis.

The presence of HER-2 over expression is associated with more aggressive forms of cancer (found in 25% to 30% of breast cancers). Therefore determination of HER-2 overexpression is a predictive factor in the therapy of breast cancer. HER-2 overexpression was shown to signify resistance to cyclophosphamide/methotrexate/5-fluoracil therapy and tamoxifen therapy. Also higher sensitivity to the high doses of anthracycline containing regimens has been observed.

Normal epithelial cells typically contain two copies of the HER-2/neu gene and express low levels of HER-2/neu receptor on the cell surface. In some cases, during oncogenic transformation the number of gene copies per cell is increased, leading to an increase in messenger Ribonucleic Acid (mRNA) transcription and a 10- to 100-fold increase in the number of HER-2/neureceptors on the cell's surface, called overexpression.

In general, the presence of HER-2/neu overexpression appears to be a key factor in malignant transformation and is predictive of a poor prognosis in breast cancer. A standard test used to measure HER-2/neu protein expression is IHC. IHC has been specifically adapted for detection of HER-2/neu protein using specific antibodies. As seen with most of the histopathological analysis, there is inter-laboratory variation in HER-2/neu overexpression scoring due to subjective measures of staining intensity and pattern. It is widely acknowledged that the ideal test for HER-2 status is one that is simple to perform, specific, sensitive, standardized, stable over time, and allows archival tissue to be assayed. At present the test that best meets these criteria is IHC.

Evaluation of HER-2/neu has become all the more important with the development of Herceptin® (i.e., trastuzamab package insert) which directly targets the HER-2/neu protein and appears to be useful in late stage metastatic adenocarcinoma of the breast. Thus, the evaluation of HER-2/neu is clinically important for at least two things; the first is, as a predictive marker for response to Herceptin® therapy and the second is, as a prognostic marker. Analysis of HER-2/neu amplification is the sole criteria for treatment with Herceptin. To summarise, accurate detection of HER-2/neu amplification is important in the prognosis and selection of appropriate therapy and prediction of therapeutic outcome.

Diagnostic methods in pathology carry out the detection, identification, quantitation and characterization of cells of interest. For example, in oncology, detection of cancer cells can be done by various methods, such as contrast enhancement by different dyes or by using a specific probe such an monoclonal antibody that reacts with component of cells of interest or by probes that are specific for nucleic acids.

IHC is a technique that detects specific antigens present in the target cells by labeling them with antibodies against them which are tagged with enzymes such as alkaline phosphatase or horseradish peroxidase (HRP) to convert a soluble colorless substrate to a colored insoluble precipitate which can be detected under the microscope. Enzyme-conjugated secondary antibodies help visualize the specific staining after adding the enzyme-specific substrate. Tissue labeled with antibodies tagged to HRP shows a brown colour deposited because of conversion of substrate of 3′,3-diaminobenzidine tetrahydrochloride (DAB) by HRP. It gets localized at the site where the marker is expressed in the cell. For example, HER-2/neu is localized at the cell membrane marking the cell membrane completely or partially. To enhance the contrast cells are counterstained with haematoxylin which stains the nuclei blue-black.

With standardization of laboratory testing and appropriate quality control in place, the reliability of IHC will be improved further. Though a more sensitive reproducible and reliable method for detection of HER-2/neu amplification at gene level is fluorescence in situ hybridization (FISH), IHC remains the most common and economical method for HER-2/neu analysis.

In the field of medical diagnostics including oncology, the detection, identification, quantification and characterization of cells of interest, such as cancer cells, through testing of biological samples is an important aspect of diagnosis. Typically, a tissue sample is prepared by staining the tissue with dyes to identify cells of interest.

Examination of biological tissues typically has been performed manually by either a lab technician or a pathologist. In the manual method, a slide prepared with a biological sample is viewed at a low magnification under a microscope to visually locate candidate cells of interest. Those areas of the slide where cells of interest are located are then viewed at a higher magnification to confirm those objects as cells of interest, such as tumor or cancer cells.

In the last few years, slides with stained biological samples are photographed to create digital images from the slides. Digital images are typically obtained using a microscope and capturing a digital image of a magnified biological sample.

A digital image typically includes an array, usually a rectangular matrix, of pixels. Each “pixel” is one picture element and is a digital quantity that is a value that represents some property of the image at a location in the array corresponding to a particular location in the image. Typically, in continuous tone black and white images the pixel values represent a “gray scale” value.

Pixel values for a digital image typically conform to a specified range. For example, each array element may be one byte (i.e., eight bits). With one-byte pixels, pixel values range from zero to 255. In a gray scale image a 255 may represent absolute white and zero total black (or visa-versa).

Color images consist of three color planes, generally corresponding to red, green, and blue (RGB). For a particular pixel, there is one value for each of these color planes, (i.e., a value representing the red component, a value representing the green component, and a value representing the blue component). By varying the intensity of these three components, all colors in the color spectrum typically may be created.

However, many images do not have pixel values that make effective use of the full dynamic range of pixel values available on an output device. For example, in the eight-bit or byte case, a particular image may in its digital form only contain pixel values that fall somewhere in the middle of the gray scale range. Similarly, an eight-bit color image may also have RGB values that fall within a range some where in middle of the range available for the output device. The result in either case is that the output is relatively dull in appearance.

The visual appearance of an image can often be improved by remapping the pixel values to take advantage of the full range of possible outputs. That procedure is called “contrast enhancement.” While many two-dimensional images can be viewed with the naked eye for simple analysis, many other two-dimensional images must be carefully examined and analyzed. One of the most commonly examined/analyzed two-dimensional images is acquired using a digital camera connected to an optical microscope.

One type of commonly examined two-dimensional digital images is digital images made from biological samples including cells, tissue samples, etc. Such digital images are commonly used to analyze biological samples including a determination of certain knowledge of medical conditions for humans and animals. For example, digital images are used to determine cell proliferate disorders such as cancers, etc. in humans and animals.

There are several problems associated with using existing digital image analysis techniques for analyzing digital images for determining know medical conditions. One problem is that existing digital image analysis techniques are typically used only for analyzing measurements of chemical compounds applied to biological samples such as groups of cells from a tissue sample. Another problem is the manual method used by pathologists is time consuming and prone to error including missing areas of the slide including tumor or cancer cells.

There have been attempts to solve some of the problems associated with manual methods for analyzing biological samples. Automated cell analysis systems have been developed to improve the speed and accuracy of the testing process. For example, U.S. Pat. No. 6,546,123 entitled “Automated detection of objects in a biological sample” that issued to McLaren, et al. on Apr. 8, 2003, includes a method, system, and apparatus are provided for automated light microscopic for detection of proteins associated with cell proliferate disorders. In a specific embodiment the McLaren invention provides an automated system for the quantitation of proteins associated with cell proliferate disorders, such as HER2/neu expression in tissue. The invention is useful to determine the over-expression of HER2 in tissue, especially breast tissue.

Another example is pending U.S. patent application No. 20030170703 entitled “Method and/or system for analyzing biological samples using a computer system” published by Piper et al. This pending U.S. Patent Application currently teaches a method and/or system for making determinations regarding samples from biologic sources. A computer implemented method and/or system can be used to automate parts of the analysis. In certain embodiments, the invention involves methods and/or systems for the estimation of gene copy number and/or detection of gene amplification in tissue samples. In particular embodiments, estimates of gene copy number can be used to accomplish or assist in diagnoses of a variety of diseases or other conditions. In certain embodiments, gene copy numbers are measured and/or estimated using one or more imaging techniques. While the invention broadly involves methods relating to measuring and/or estimating biologic characteristics of samples, the invention may be further understood by considering as an example the problem of determining whether a particular breast cancer is likely to respond to treatments targeting HER-2/neu gene overexpression. It is currently believed that one method of determining if a breast cancer will respond to treatments targeting HER-2/neu, such as Herceptin is by determining and/or estimating HER-2/neu copy numbers in cells that are identified as invasive cancer cells.

However, these attempts still do not solve all of the problems with automated biological analysis systems that have been developed to improve the speed and accuracy of the testing process. Thus, it is desirable to provide an automated biological sample analysis system that not only provides automated analysis of biological samples based on analyzing an intensity of a chemical or biological marker, but also on the morphological features of the biological sample.

### SUMMARY OF THE INVENTION

In accordance with preferred embodiments of the present invention, some of the problems associated with automated biological sample analysis systems are overcome. A method and system for automatically determining diagnostic saliency of digital images is presented.

Luminance parameters (e.g., intensity, etc. from a digital image of a biological sample (e.g., tissue cells) to which a chemical compound (e.g., a marker dye) has been applied are automatically analyzed and corrected if necessary. Morphological parameters (e.g., cell membrane, cell nucleus, mitotic cells, etc.) from individual components within the biological sample are automatically analyzed on the digital image. A medical conclusion (e.g., a medical diagnosis or medical prognosis) is automatically determined from the analyzed luminance and morphological parameters. The method and system may improve automated analysis of digital images including biological samples such as tissue samples and aid the diagnosis or prognosis of diseases (e.g., human cancer diagnosis or prognosis).

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1 is a block diagram illustrating an exemplary automated biological sample analysis processing system;

FIG. 2 is a flow diagram illustrating an exemplary method for automated biological sample analysis;

FIG. 3 is a flow diagram illustrating an exemplary method for automated biological sample analysis;

FIGS. 4A and 4B are a block diagram illustrating the effect of contrast modification of a digital image;

FIGS. 5A and 5B are block diagrams illustrating a histogram of luminosity values of an original and a modified digital image respectively;

FIG. 6 is block diagram illustrating additional details of FIG. 5B;

FIG. 7 is a block diagram illustrating a threshold image of the digital image illustrated in FIG. 6A;

FIG. 8 is a block diagram illustrating a color corrected digital image corresponding to the original image illustrated in FIG. 6A;

FIG. 9 is a block diagram illustrating digital images of cells including 0+ through 3+ HER-2/neu grade scoring.

FIG. 10 is a block diagram illustrating a sample cell nucleus with incomplete classified membrane rings;

FIG. 11 is a block diagram illustrating the same cell nucleus of FIG. 10 with complete classified membrane rings;

FIGS. 12A, 12B and 12C are a block diagram illustrating a sample digital image with varying degrees of HER-2/neu amplification;

FIG. 13 is a flow diagram illustrating an automated method for biological sample analysis;

FIGS. 14A, 14B and 14C are a flow diagram illustrating an automated method for measuring morphological features in digital images; and

FIG. 15 is a flow diagram illustrating an automated method for biological sample analysis.

FIG. 16A is a block diagram illustrating an original sample digital image;

FIG. 16B is a block diagram illustrating the digital image of FIG. 16A with plural luminance parameters adjusted.

FIG. 16C is a block diagram illustrating the digital image of FIG. 16 with plural epithelial areas identified.

FIG. 16D is a block diagram illustrating the digital image of FIG. 16A with plural nuclei identified.

FIG. 16E is a block diagram illustrating the digital image of FIG. 16A with plural membranes detected.

FIG. 16F is a block diagram illustrating the digital image of FIG. 16A with plural cell classifications based on plural membranous patterns, nuclei and membranes.

FIG. 17 is a flow diagram illustrating a method for identification of individual morphological components from one or more adjusted areas of interest within an digital image;

FIG. 18 is a flow diagram illustrating a method for identification of cell nuclei from one or more adjusted areas of interest within an digital image;

FIGS. 19A and 19B are a flow diagram illustrating a method for classification of cell nuclei according to a pre-defined classification scheme;

FIGS. 20A and 20B are a flow diagram illustrating a method for classification of cell chromatin pattern according to a pre-defined classification scheme;

FIG. 21 is a flow diagram illustrating a method for identification of cell nucleolus from one or more adjusted areas of interest within an digital image;

FIG. 22 is a flow diagram illustrating a method for identification of mitotic cells from one or more adjusted areas of interest within an digital image;

FIGS. 23A and 23B are a flow diagram illustrating a method for detecting boundary pixels of an identified nucleus;

FIG. 24 is a flow diagram illustrating a method for counting individual biological components from one or more adjusted areas of interest within a digital image;

FIG. 25 is a flow diagram illustrating a method for quantification of individual biological components and tissue sample based on one or more adjusted areas of interest within a digital image;

FIG. 26 is a flow diagram illustrating a method for quantifying membrane stain values in identified cells in areas of interest within a digital image; and

FIG. 27 is a block diagram illustrating an exemplary flow of data in the automated biological sample analysis processing system.

### DETAILED DESCRIPTION OF THE INVENTION

### Exemplary Biological Sample Analysis System

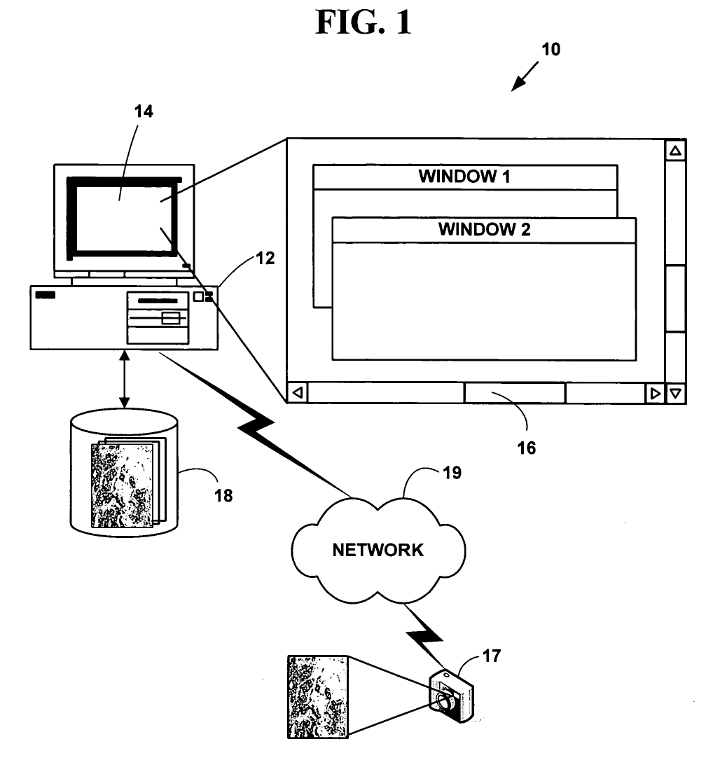

FIG. 1 is a block diagram illustrating an exemplary biological sample analysis processing system 10. The exemplary biological sample analysis processing system 10 includes one or more computers 12 with a computer display 14 (one of which is illustrated). The computer display 14 presents a windowed graphical user interface (“GUI”) 16 with multiple windows to a user. The present invention may optionally include a microscope or other magnifying device (not illustrated in FIG. 1) and/or a digital camera 17 or analog camera. One or more databases 18 (one or which is illustrated) include biological sample information in various digital images or digital data formats. The databases 18 may be integral to a memory system on the computer 12 or in secondary storage such as a hard disk, floppy disk, optical disk, or other non-volatile mass storage devices. The computer 12 and the databases 18 may also be connected to an accessible via one or more communications networks 19.

The one or more computers 12 may be replaced with client terminals in communications with one or more servers, or with personal digital/data assistants (PDA), laptop computers, mobile computers, Internet appliances, one or two-way pagers, mobile phones, or other similar desktop, mobile or hand-held electronic devices.

The communications network 19 includes, but is not limited to, the Internet, an intranet, a wired Local Area Network (LAN), a wireless LAN (WiLAN), a Wide Area Network (WAN), a Metropolitan Area Network (MAN), Public Switched Telephone Network (PSTN) and other types of communications networks 19.

The communications network 19 may include one or more gateways, routers, or bridges. As is known in the art, a gateway connects computer networks using different network protocols and/or operating at different transmission capacities. A router receives transmitted messages and forwards them to their correct destinations over the most efficient available route. A bridge is a device that connects networks using the same communications protocols so that information can be passed from one network device to another.

The communications network 19 may include one or more servers and one or more web-sites accessible by users to send and receive information useable by the one or more computers 12. The one ore more servers, may also include one or more associated databases for storing electronic information.

The communications network 19 includes, but is not limited to, data networks using the Transmission Control Protocol (TCP), User Datagram Protocol (UDP), Internet Protocol (IP) and other data protocols.

As is know in the art, TCP provides a connection-oriented, end-to-end reliable protocol designed to fit into a layered hierarchy of protocols which support multi-network applications. TCP provides for reliable inter-process communication between pairs of processes in network devices attached to distinct but interconnected networks. For more information on TCP see Internet Engineering Task Force (ITEF) Request For Comments (RFC)-793, the contents of which are incorporated herein by reference.

As is know in the art, UDP provides a connectionless mode of communications with datagrams in an interconnected set of computer networks. UDP provides a transaction oriented datagram protocol, where delivery and duplicate packet protection are not guaranteed. For more information on UDP see IETF RFC-768, the contents of which incorporated herein by reference.

As is known in the art, IP is an addressing protocol designed to route traffic within a network or between networks. IP is described in IETF Request For Comments (RFC)-791, the contents of which are incorporated herein by reference. However, more fewer or other protocols can also be used on the communications network 19 and the present invention is not limited to TCP/UDP/IP.

The one or more database 18 include plural digital images of biological samples taken with a camera such as a digital camera and stored in a variety of digital image formats including, bit-mapped, joint pictures expert group (JPEG), graphics interchange format (GIF), etc. However, the present invention is not limited to these digital image formats and other digital image or digital data formats can also be used to practice the invention.

The digital images are typically obtained by magnifying the biological samples with a microscope or other magnifying device and capturing a digital image of the magnified biological sample (e.g., groupings of plural magnified cells, etc.).

The term “sample” includes, but is not limited to, cellular material derived from a biological organism. Such samples include but are not limited to hair, skin samples, tissue samples, cultured cells, cultured cell media, and biological fluids. The term “tissue” refers to a mass of connected cells (e.g., central nervous system (CNS) tissue, neural tissue, or eye tissue) derived from a human or other animal and includes the connecting material and the liquid material in association with the cells. The term “biological fluid” refers to liquid material derived from a human or other animal. Such biological fluids include, but are not limited to, blood, plasma, serum, serum derivatives, bile, phlegm, saliva, sweat, amniotic fluid, and cerebrospinal fluid (CSF), such as lumbar or ventricular CSF. The term “sample” also includes media containing isolated cells. The quantity of sample required to obtain a reaction may be determined by one skilled in the art by standard laboratory techniques. The optimal quantity of sample may be determined by serial dilution.

An operating environment for the devices biological sample analysis processing system 10 include a processing system with one or more high speed Central Processing Unit(s) (“CPU”), processors and one or more memories. In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed,” “CPU-executed,” or “processor-executed.”

It will be appreciated that acts and symbolically represented operations or instructions include the manipulation of electrical signals by the CPU or processor. An electrical system represents data bits which cause a resulting transformation or reduction of the electrical signals or biological signals, and the maintenance of data bits at memory locations in a memory system to thereby reconfigure or otherwise alter the CPU's or processor's operation, as well as other processing of signals. The memory locations where data bits are maintained are physical locations that have particular electrical, magnetic, optical, or organic properties corresponding to the data bits.

The data bits may also be maintained on a computer readable medium including magnetic disks, optical disks, organic memory, and any other volatile (e.g., Random Access Memory (“RAM”)) or non-volatile (e.g., Read-Only Memory (“ROM”), flash memory, etc.) mass storage system readable by the CPU. The computer readable medium includes cooperating or interconnected computer readable medium, which exist exclusively on the processing system or can be distributed among multiple interconnected processing systems that may be local or remote to the processing system.

### Exemplary Automated Biological Sample Analysis Method

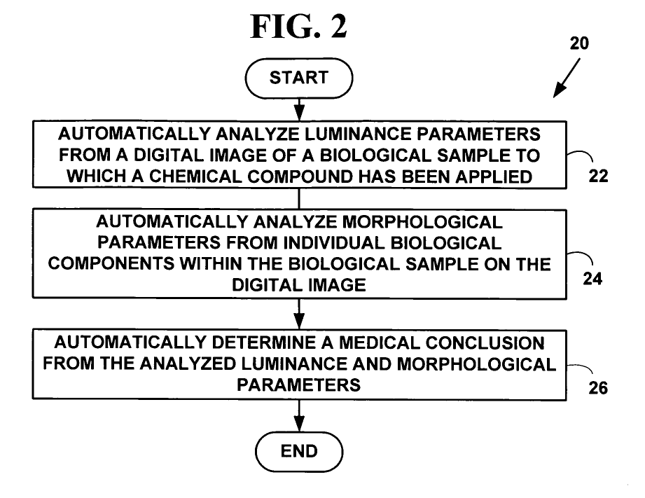

FIG. 2 is a flow diagram illustrating a Method 20 for automated biological sample analysis. At Step 22, luminance parameters from a digital image of a biological sample to which a chemical compound has been applied are automatically analyzed and corrected if necessary. At Step 24, morphological parameters from individual components within the biological sample are automatically analyzed on the digital image. At Step 26, a medical conclusion is automatically determined from the analyzed luminance and morphological parameters.

Method 20 is illustrated with one exemplary embodiment. However, the present invention is not limited to such an embodiment and other embodiments can also be used to practice the invention. In such an exemplary embodiment at Step 22, luminance parameters such as intensity, from a digital image of a biological sample (e.g., tissue cells) to which a chemical compound (e.g., a marker dye) has been applied are automatically analyzed and corrected if necessary.

At Step 24, morphological parameters (e.g., cell membrane, cell nucleus, etc.) from individual components within the biological sample are automatically analyzed on the digital image. Step 24, includes, but is not limited to, automatically identifying and analyzing individual morphological components from a biological tissue sample within a digital image (e.g., cell nuclei, cell membrane, cell chromatin pattern, mitotic cells, epithelial area, fibrin, etc. and other material in the tissue sample).

At Step 26, a medical conclusion (e.g., a medical diagnosis or a medical prognosis) is automatically determined from the automatically analyzed luminance and morphological parameters.

Method 20 may be specifically applied by pathologists and other medical personnel to analyze a tissue sample for cancer cells and make a medical diagnosis. However, the present invention is not limited to such an application and Method 20 may be used for other purposes.

Method 20 may be used for automatically determining diagnostic saliency of digital images for mammalian cancer cells. In one embodiment, the method is used for automatically determining diagnostic saliency of digital images for human cancer cells includes using plural filters (e.g., one or more of Equations 1-15) for evaluating digital images. Each filter is designed to improve a specific type of medical diagnostic finding.

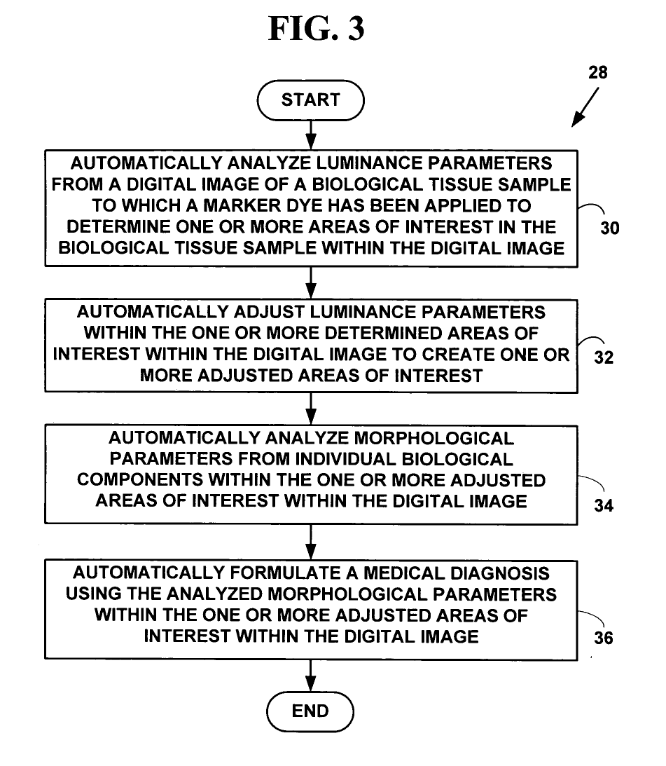

FIG. 3 is a flow diagram illustrating a Method 28 for automated biological sample analysis. At Step 30, luminance parameters from a digital image of a biological tissue sample to which a marker dye has been applied are automatically analyzed to determine one or more areas of interest in the biological tissue sample within the digital image. At Step 32, luminance parameters within the one or more determined areas of interest within the digital image are automatically adjusted to create one or more adjusted areas of interest. At Step 34, morphological parameters from individual biological components within the one or more adjusted areas of interest within the digital image are automatically analyzed. At Step 36, a medical diagnosis is automatically formulated using the analyzed morphological parameters within the one or more adjusted areas of interest within the digital image.

In one embodiment, Method 28 further comprises the steps of: automatically formulating a medical diagnosis using the analyzed morphological parameters within the one or more adjusted areas of interest within the digital image and one or more diagnostic knowledge records from a knowledge database; and automatically saving the formulating medical diagnosis in the knowledge database to create additional diagnostic knowledge.

Method 28 is illustrated with one exemplary embodiment. However, the present invention is not limited to such an embodiment and other embodiments can also be used to practice the invention. In such an exemplary embodiment, the marker dye includes IHC staining or other types of staining of human tissue samples including plural human cells. The human tissue sample may potentially include one or more human cancer cells. One or more digital images are created by photographing the plural human cells to which the H/E staining has been applied through an optical microscope with a desired magnification. However, if other types of maker dyes or stains IHCs are used then the various cell components of the plural human cells would typically comprise other colors and intensities. These cell components are areas of interest automatically determined at Step 30.

Digital images captured through an optical microscope resemble a view a human pathologist gets through optical system of a microscope. However, a human pathologist based on his/her experience is in a position to easily distinguish between nuclei, cytoplasm, red blood cells, membranous pattern and fibrin, even-though there are variations in staining, variations in illumination across slide. A human pathologist has experience and knowledge of the domain of pathological analysis of tissue cells to distinguish between the various cellular components.

In one embodiment of the invention, Method 28 is used for automatic HER-2/neu grading. In general, HER-2/neu grading is manually done in two steps. A specimen slide is scanned at low magnification to detect cells of interest, in this case potential cancer cells with positive brown staining for HER-2 where the specimen has been treated with IHC staining and counter stained with haematoxylin. The same specimen is then viewed at higher magnification and the potential cancer cells confirmed as positive cells. HER-2/neu scoring is then completed depending upon the staining.

A pathologist thus manually positions a microscope and counts cells. This manual procedure is time intensive, can introduce errors and lead to improper diagnosis of carcinomas. In one embodiment of the invention, an automated cell analysis systems has been developed to improve the speed and accuracy of the, HER-2/neu grading process using Method 28.

In one embodiment of the invention, to obtain a HER-2/neu grade based on a digital image of a biological tissue sample includes, but is not limited to, automatically determining a “segmentation” and a “grading” of a biological tissue sample in a digital image.

### Segmentation Method

“Segmentation” includes segmenting individual morphological components from the biological tissue sample within the digital image (e.g., cell nuclei, and cell membrane from the cytoplasm, fibrin and other material in the tissue sample, mitotic cells, etc.).

Returning to FIG. 3 at Step 32, luminance parameters within the one or more determined areas of interest within the digital image are automatically adjusted to create one or more adjusted areas of interest. In one embodiment of the invention, an automatic segmentation method practiced at Step 32 achieves the same results as completed by a human pathologist but in an automated manner. However, the present invention is not limited to this automatic segmentation method and other methods can be practiced at Step 32.

The segmentation method includes, but is not limited to, at least three distinct steps: (1) Contrast modification of a digital image based on image statistics; (2) Thresholding a contrast modified digital image to obtain selected pixels; and (3) Correcting color components of the selected pixels. However, the present invention is not limited to these steps and more, fewer or other steps can also be used to practice the invention.

“Contrast” in a digital image is referred to the difference in luminosity level between any two given pixels. Luminosity at a given pixel is computed from Red, Green and Blue components of a given color digital image using the formula illustrated in

Equation 1:
Y=XG+YR+ZB, (1)

where R,G and B are Red, Green and Blue color component values of a pixel respectively and X, Y and Z are predetermined constant values.

In one exemplary embodiment of the invention the predetermined constant values include, but are limited to, for example, X=0.59, Y=0.29 and Z=0.12. However, the present invention is not limited to such an exemplary embodiment and other values can be used for the X, Y and Z constants.

Contrast modification: A digital image is considered “high contrast” if its luminosity levels range from a minimum value (e.g., zero) to a maximum value (e.g., 255). In the case of low contrast images, this range (e.g., 0-255) could be as small as 50, for example, or range from 100 to 150. In the case of high contrast images, the pixels belonging to nuclei and membrane typically have a low luminosity, cytoplasm has a moderate luminosity and vacuoles have a highest luminosity. Contrast modification helps improve low contrast images to aid automated analysis. Contrast modification is used such that dark pixels become even darker and brighter pixels maintain at least a same level of initial brightness.

Equations 2, 3 and 4 are used to compute modified Red, Green and Blue component values of a pixel in a digital image:
R′=(R*(M+D)/K)+(X−M) (2)
G′=(G*(M+D)/K)+(Y−M) (3)
B′=(B*(M+D)/K)+(Z−M), (4)
where K, Y, Y and Z are predetermined constant values and R′, G′ and B′ are modified red (R), green (G) and blue (B) components of pixel respectively. Mean (M) and standard deviation (D) of a luminosity histogram are computed using standard equations to calculate a mean and standard deviation known in the statistical arts.

With respect to contrast modification of digital images, “mean” is used a measure of average brightness and “standard deviation” is used a measure of contrast. A data checker verifies that R′, G′ and B′ values remain within allowed bounds for the particular color space.

In one exemplary embodiment of the invention, include, but is not limited to, for example, K=100 and X, Y and Z all equal to 128. However, the invention is not limited to such values and other values can also be used for the predetermined constants, K, X, Y and Z and the constants need not be all equal to the same value.

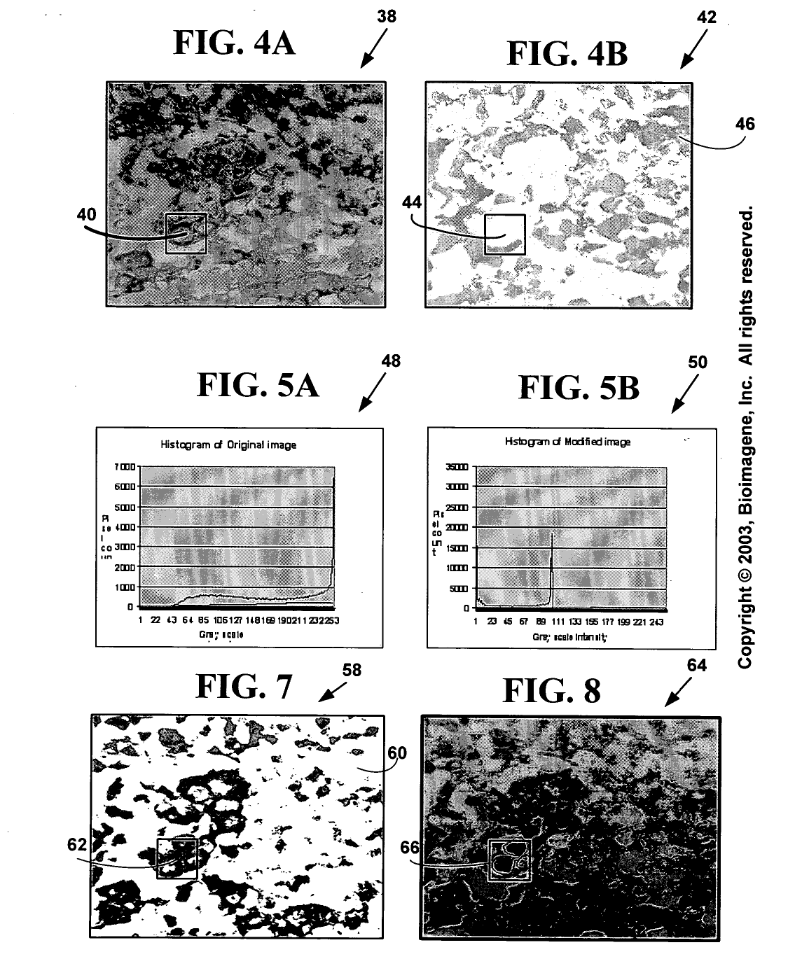

FIG. 4 illustrates the effect of contrast modification. FIG. 4A is a block diagram 38 illustrating an original digital image of plural human tissue cells before contrast modification. An area of interest is illustrated at 40. FIG. 4B is a block diagram 42 illustrating the same digital image modified with contrast modification at Step 32. One or more adjusted areas of interest (e.g., area of interest 40) including plural cell components and potential cancer cells are adjusted to look lighter 44 with areas not of interest adjusted to be darker 46.

Thresholding: A thresholding operation is carried out on a modified digital image. A luminosity histogram grayscale of values of the modified digital image is computed using Equation 1. However, the present invention is not limited to such an embodiment, and other embodiments can also be used to provide thresholding.

As is known in the arts, a “grayscale” is a sequence of shades ranging from black through white, used in computer graphics to add detail to digital images. Grayscales are also used to represent a color image on a monochrome output device or analyze a color image. Like the number of colors in a color image, the number of shades of gray depends on the number of bits stored per pixel.

FIG. 5 is a block diagram illustrating histograms of luminosity values computed from digital images. FIG. 5A is a block diagram 48 illustrating a histogram of grayscale luminosity values from the original digital image 38 of FIG. 4A. FIG. 5B is a block diagram 50 illustrating a histogram of grayscale luminosity values from the modified digital image 42 of FIG. 4B.

The luminosity histogram of modified digital image 42 has typically has two peaks with a valley in between them. For example, there is a peak at about a value of one and a peak at about a value of about 111 of gray scale intensity in FIG. 5B.

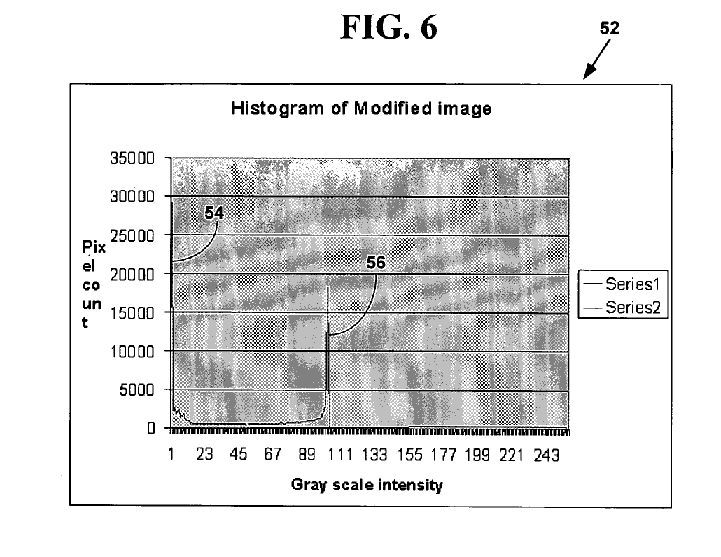

FIG. 6 is a block diagram 52 illustrating additional details of the histogram of grayscale luminosity values of a modified digital image of FIG. 5B. The histogram of luminosity values of a contrast modified image typically always exhibit a bimodal nature as is illustrated in FIG. 6 (e.g., first peak 54 at about one and second peak 56 about 111 of grayscale intensity).

The first peak 54 is observed with lower grayscale value (e.g., one) should correspond to the pixels of a biological tissue component (e.g., nuclei, membrane, etc.) The second peak 56 observed from a highest grayscale value (e.g., 111) corresponds to a background component (e.g., vacuoles, cytoplasm, etc.). Any pixel with a grayscale value less than or equal to a maximum first peak value typically belongs to objects of interest (e.g., potential cancer cells).

FIG. 7 is a block diagram 58 illustrating a thresholded image of the modified digital image illustrated in FIG. 4B. In FIG. 7, a background 60 is a light color (e.g., colored white) while keeping the color composition of the area of interest (e.g., selected cell components) are a darker color 62.

Correcting: Correction of color components of selected cells is necessary in some digital images with low contrast or digital images with some color background. For example, it is known that objects in areas of interest, such as cancer cells, nuclei are blue in color when stained with H/E staining. Therefore, blue color values are emphasized or corrected by multiplying with a correction factor C_{f} illustrated in Equation 5. However, the present invention is not limited to blue color values and other color values can also be used to practice the invention with other types of stains.
C_{f}=(Mean of a first color plane)/(mean of a second color plane) in the original image (5)

In one embodiment of the invention, for example, the first color plane is a blue plane and the second color plane is a red plane. However, the present invention is not limited to this embodiment and other correction factors can also be used and other color planes and combinations thereof can also be used to calculate the correction factor. For example, if a biological tissue sample when treated with other than H/E staining, then nuclei or other cell components may appear as a different color other than blue and the correction factor C_{f} would be calculated using other than the red and blue color planes.

In one embodiment of the invention, a blue color plane component of all pixels in objects of interest (e.g., potential cancer cells) is multiplied by C_{f}, if C_{f} is greater than one. If C_{f} is less than or equal to one, no color correction is applied. Pixels belonging to a background in the digital image are kept unchanged irrespective of C_{f} value. In this embodiment, as a result of the C_{f} correction there will be increase in the difference of blue components between pixels belonging to the cells and those belonging to the background. However, the present invention is not limited to this embodiment, and other types of color correction can also be applied.

FIG. 8 is a block diagram 64 illustrating a color corrected digital image corresponding to the original image 38 illustrated in FIG. 4A. Objects such as potential cancer cells appear with sharper or additional contrast in areas of interest 66 in FIG. 8 compared to the area of interest 40 in the original image 38 (FIG. 4A).

Returning to FIG. 3 at Step 34, morphological parameters from individual biological components within the one or more adjusted areas of interest within the digital image are automatically analyzed.

For example, to obtain a HER-2/neu grade based on a digital image includes determining a grading. “Grading: includes computing a grade (e.g., a HER-2/neu grade) based on morphological parameters from individual biological components within the one or more adjusted areas of interest within the digital image (e.g., continuity of membranous rings around nuclei, etc.).

Human pathologists make use of several parameters that are typically hard to quantify for grading purposes. For example, it is typically difficult to specify terms like “at most faint”, “equivocal”, “moderate intensity” into an automated analysis method. In the present invention, a deterministic approach that approximates human pathologist's grading is used via an automated method.

Genetic and other tests detect the presence of specific genes that are associated with the suppression of tumor growth, such as p53, or oncogenes (i.e., cancer promoting genes), such as HER-2/neu. The HER-2/neu protein, made by the mutated gene, can be measured and reported as one to three plus format, depending on the overexpression of the protein.

In one embodiment of the invention, a medical diagnosis based on HER-2/neu overexpression scoring (e.g., at Step 36), for example, is done using the following system: “1+,” those tumors showing at most faint, equivocal, and incomplete membranous staining; “2+,” unequivocal, complete membranous pattern, with moderate intensity; and “3+,” those tumors that showed areas of strong, membranous pattern. The one to three plus format applied within the one or more adjusted areas of interest within the digital image at Step 34 to automatically formulate a medical diagnosis at Step 36.

Table 1 illustrates examples of HER-2/neu scoring.

TABLE 1

| | Amount of overexpression | HER-2/neu |
|---|---|---|
| No. | in percentage of cells | Grade |
| 1 | <5 | 0+ |
| 2 | >5 and <15 | 1+ |
| 3 | >15 and <75 | 2+ |
| 4 | >75 | 3+ |

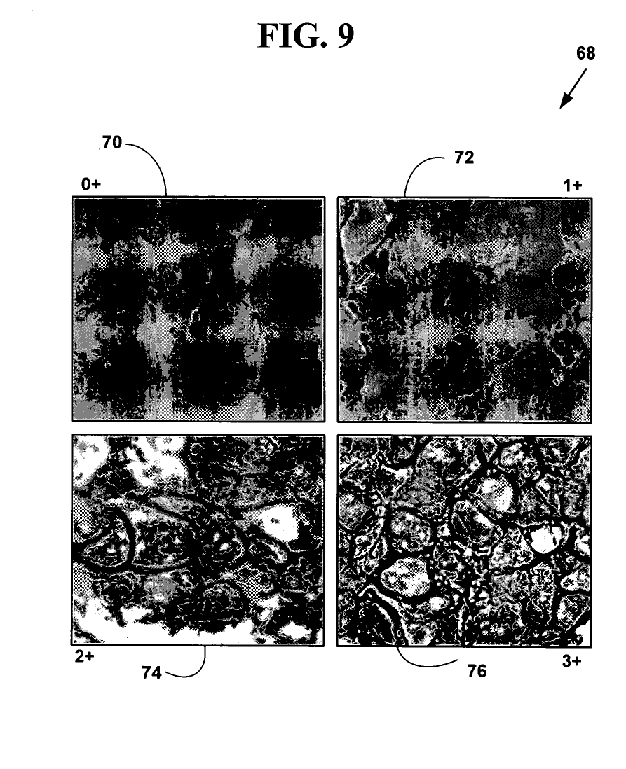

FIG. 9 is a block diagram 68 illustrating digital images of cells including 0+, 1+, 2+ and 3+ scoring 70, 72, 74, 76 respectively as is illustrated in Table 1.

Returning to FIG. 3 at Step 34 a deterministic approach is used that includes, but is not limited to, at least three steps: (1) Broad classification: broadly classifying into two or more groups based on the presence of pre-determined pixels using the analyzed morphological parameters within the one or more adjusted areas of interest within the digital image; (2) Detection: detecting one or more predetermined analyzed morphological parameters within the one or more adjusted areas of interest within the digital image; and (3) Final grading: preparing a final grading based on a completeness of the one or more predetermined analyzed morphological parameters. However, the present invention is not limited to these steps and more, fewer or other steps can also be used to practice the invention.

Broad classification: In one embodiment, it is observed that pixels on the digital image belonging to a cell membrane are of brown or dark red in color as stained with IHC and pixels belonging to a cell nucleus are blue or dark blue in color when stained with Haematoxylin. Brown pixels tend to form rings around blue nucleus. For HER-2/neu scoring a first level classification is completed by grouping 0+, and 1+ into a first group and 2+ and 3+ into a second group based on the ratio of brown pixels versus blue pixels. A pixel is stained brown if a red component of the same pixel is greater than the blue component. However, the present invention is not limited to this embodiment. Other stains and/or IHCs will produce cell components highlighted with other colors and other color components of pixels will then be used to practice the invention.

For every cell pixel (e.g., selected as result of the segmentation method described above), the pixel is counted if it is a stained (e.g., brown in color) cell pixel, if first color component (e.g., Red) of a pixel >(C_{f}*a second component of pixel (e.g., Blue)). Otherwise it is counted as non-stained cell pixel.

A stained pixel percentage=stained cell pixels/(stained cell pixels+non stained pixels). If the stained pixel percentage exceeds a pre-determined percentage (e.g., >15%), then the image belongs to second group (e.g., 2+ or 3+), otherwise it belongs to a first group (e.g., 0+ or 1+). Further division of the first and second groups is also done based on the stained pixel percentage.

If a stained Pixel percentage less than first pre-determined percentage (e.g., 5%) it is graded as a first grade (e.g., 0+). If a stained Pixel percentage less than a second pre-determined percentage (e.g., 15%) but more than the first pre-determined percentage (e.g., 5%) it is graded as a second grade (e.g., 1+).

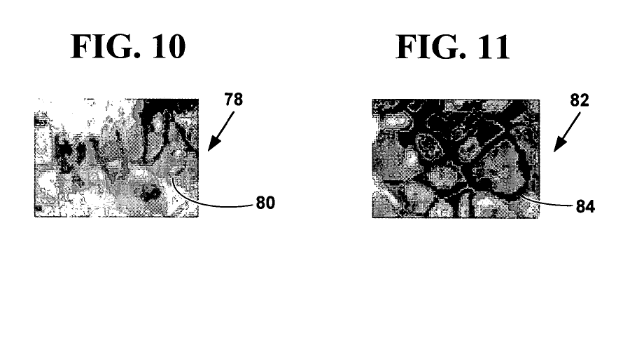

FIG. 10 is a block diagram 78 illustrating a sample cell nucleus with incomplete classified membrane rings 80.

FIG. 11 is a block diagram 82 illustrating the same cell nucleus of FIG. 10 with complete classified membrane rings 84.

Detection: Detecting membrane intensity for HER-2/neu scoring. Two parameters are checked to decide between 2+ and 3+ grades. FIG. 10 illustrates incomplete (or partial) rings 80 of cell membrane staining. FIG. 11 illustrates complete rings 84 of cell membrane staining. A first parameter used is detection of clear and complete membrane ring around nucleus and a second parameter is an intensity of the membranous pattern. These parameters are examined from a geometrical point of view. Cells are supposed to be spherical in nature. Membranes enclose these cells. Therefore, ideally any cross section of a cell should be a blue circle enclosed by a thin brown ring. This is true if all of the individual cells are separated, which is not realistic. As a greater number of cells get pressed together (e.g., in a tumor), they loose their circular shape and vary in shape. The thickness of the brown layer between two adjacent cells will be at the most equal to sum of the thickness of both cells. This results in dark brown pixels with blue pixels on a direction perpendicular to the point of contact.

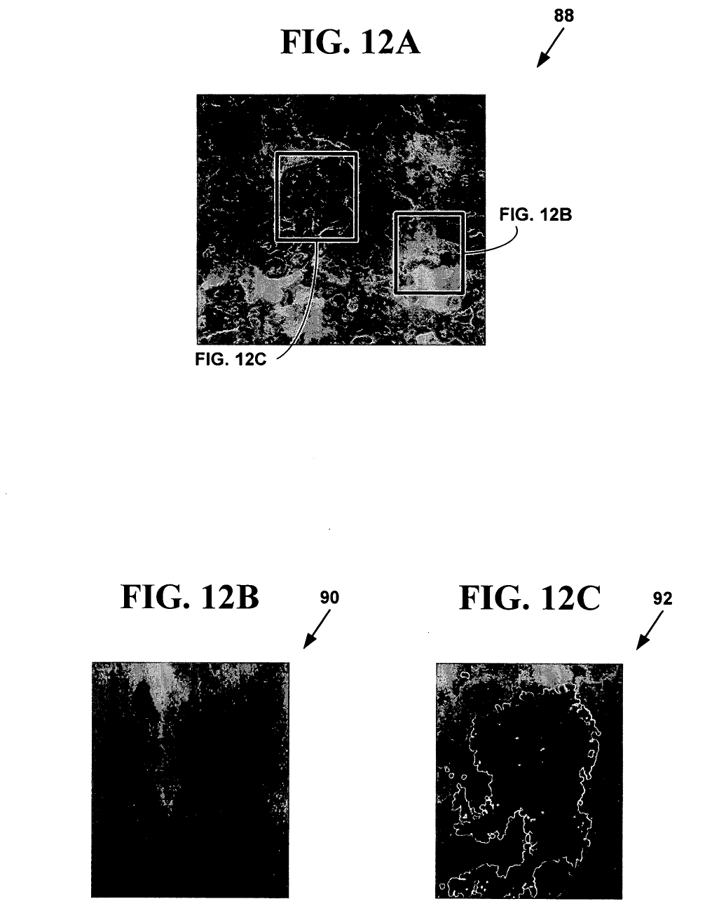

FIG. 12 illustrates a sample digital image with varying HER-2/-neu amplification is illustrated. This image has a clear membranous pattern (e.g., 3+ in FIG. 12C) and epithelial cells without any membrane (e.g., 0+ in FIG. 12B).

FIG. 12A is a block diagram 88 illustrating an original sample digital image. FIG. 12B is a block diagram 90 illustrating a 0+ part of the image of FIG. 12A. FIG. 12C is a block diagram 92 illustrating a 3+ part of the original image of FIG. 12A.

As is illustrated in FIG. 12C, even though several cells are pressed together, the ratio of perimeter over area of nucleus part remains same. The only change is in the shape from circular to a closed curve. A perimeter represents the brown ring length, which is divided amongst two adjacent cells and area can gives a diameter of nucleus.

When biological tissues are stained, morphological components within individual biological components often include two or more colors that are used to identify the morphological components. For example, using H/E staining, cell membranes stain brown and other cell components stain blue.

FIG. 12C and Equation 7 illustrates this point. If a membrane ring is unit width, then
length of complete cell ring first constant*a number of stained cell pixels of a first color=Pi*r*d, (7)

where the “first constant” is a pre-determined number (e.g., 0.5), “r” is a radius of the enclosed nucleus and “d” is a thickness of the ring and “Pi” is the constant “3.1415927 . . . ”.

In one embodiment of the invention, the complete cell ring is a complete brown cell ring and the number of stained pixels is a number of stained brown pixels. In such an embodiment, the cells from the biological tissue sample have been stained with IHC staining and conunter stained with haematoxylin. However, the present invention is not limited to brown colored cells and if other stains are used, then cells stained with other colors can used in Equation 7 to practice the invention.

A radius of a nucleus can be calculated from the number of pixels of a second color as illustrated by Equation 8.
Number of enclosed pixels of second color=area of nucleus=Pi*r*d, (8)

where “d” is one unit for grade 2+and it is two more units for 3+grade images, “r” radius (e.g., about 15 units corresponding to cell size of about ten microns) at typical magnification (e.g., 40×) in an optical microscope.

In one embodiment of the invention, the second color includes blue colored pixels of cells stained with IHC. However, the present invention is not limited to blue colored cells and if other stains are used, then cells stained with other colors can used in Equation 8 to practice the invention.

In essence, for IHC staining by calculating a ratio of brown pixels over blue pixels enclosed in these brown rings an accurate estimation of membranous pattern is obtained.

The pixels on membranous mesh are determined based on an intensity in a segmented image. A membrane pixel will have lower intensity value in a blue plane than its two neighbors in any one of four selected directions (e.g., zero, 45, 90 and 135 degrees).

At Step 34, morphological parameters are automatically analyzed with a Final Grading used at Step 36. Final grading: Final grading for HER-2/neu scoring includes identification of fully stained cells or cells with a complete membrane ring. All non mesh pixels are labeled then regions bigger than normal size are ignored. A first grading ratio (e.g., about 4% of the image size) is used to determine if a region is large or not.

What remains are regions of full stained cells and membranous patterns. A ratio of mesh over the stained cell area is used to decide upon a final grade. If a ratio of mesh pixels/stained cell pixels is less than a second grading ratio (e.g., about 7%), then it is graded as a second grade (e.g., 2+). Otherwise it is graded as a third grade (e.g., 3+). A constant grading ratio of about 7% was derived from Equations (7) and (8). However, the present invention is not limited to this constant and other constants and grading ratios can also be used. At Step 36, a medical diagnosis using the analyzed morphological parameters within the one or more adjusted areas of interest within the digital image is formulated.

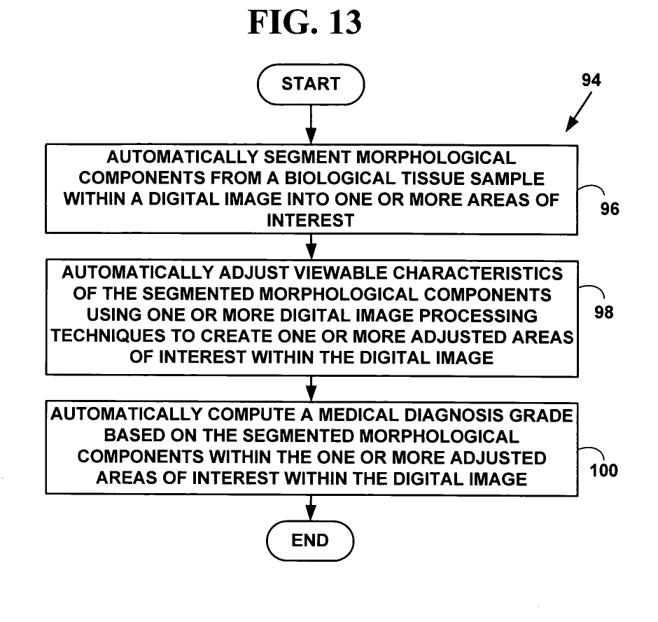

FIG. 13 is a flow diagram illustrating a Method 94 for automated method for biological sample analysis. At Step 96 morphological components from a biological tissue sample within a digital image are automatically segmented into one or more areas of interest. At Step 98, viewable characteristics of the segmented morphological components are automatically adjusted using one or more digital image processing techniques to create one or more adjusted areas of interest within the digital image. At Step 100, a medical diagnosis grade is automatically formulated based on the segmented morphological components within the one or more adjusted areas of interest within the digital image.

Method 94 is illustrated with one exemplary embodiment. However, the present invention is not limited to such an embodiment and other embodiments can also be used to practice the invention. In such an exemplary embodiment, the marker dye includes H/E staining of human tissue samples including plural human cells.

In such an exemplary embodiment at Step 96, cell nuclei and cell membranes are automatically separated from cytoplasm, fibrin and other components in the biological tissue sample within the digital image. At Step 98, viewable characteristics of the segmented morphological components are automatically adjusted by modifying a contrast of the digital image based on statistics collected from the digital image to created a contrast modified digital image; thresholding the contrast modified digital image to obtain a plural thresholded pixels in the one or more areas of interest; and correcting color components of the plurality of thresholded pixels within the one or more areas of interest in the contrast modified digital image to create one or more adjusted areas of interest within the digital image. At Step 100, a medical diagnosis grade (e.g., a HER-2/neu grade) is automatically formulated based on continuity of cell membranous rings around cell nuclei within the one or more adjusted areas of interest within the digital image.

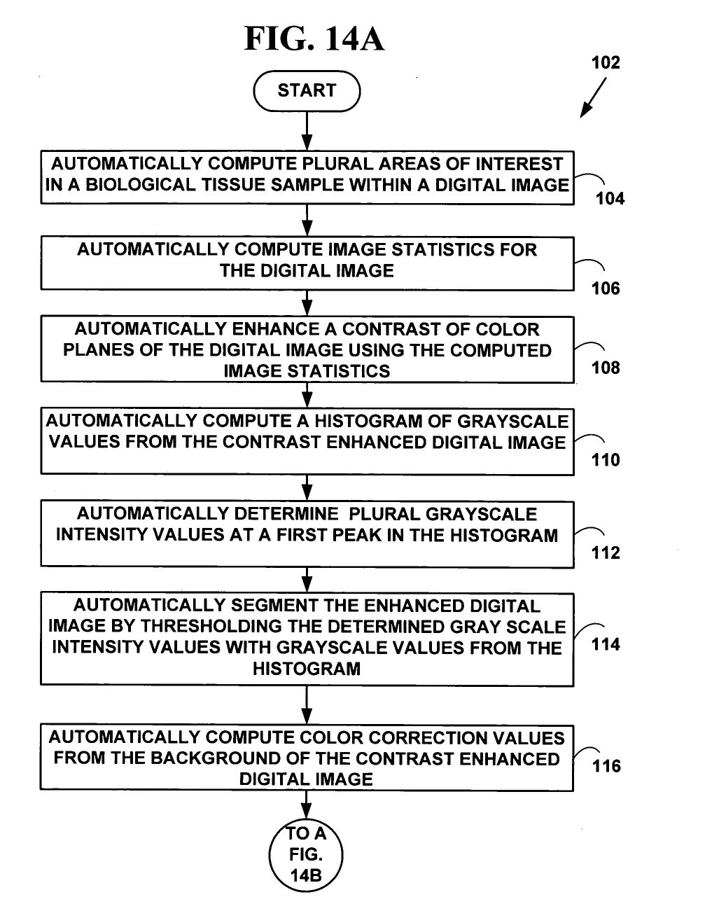

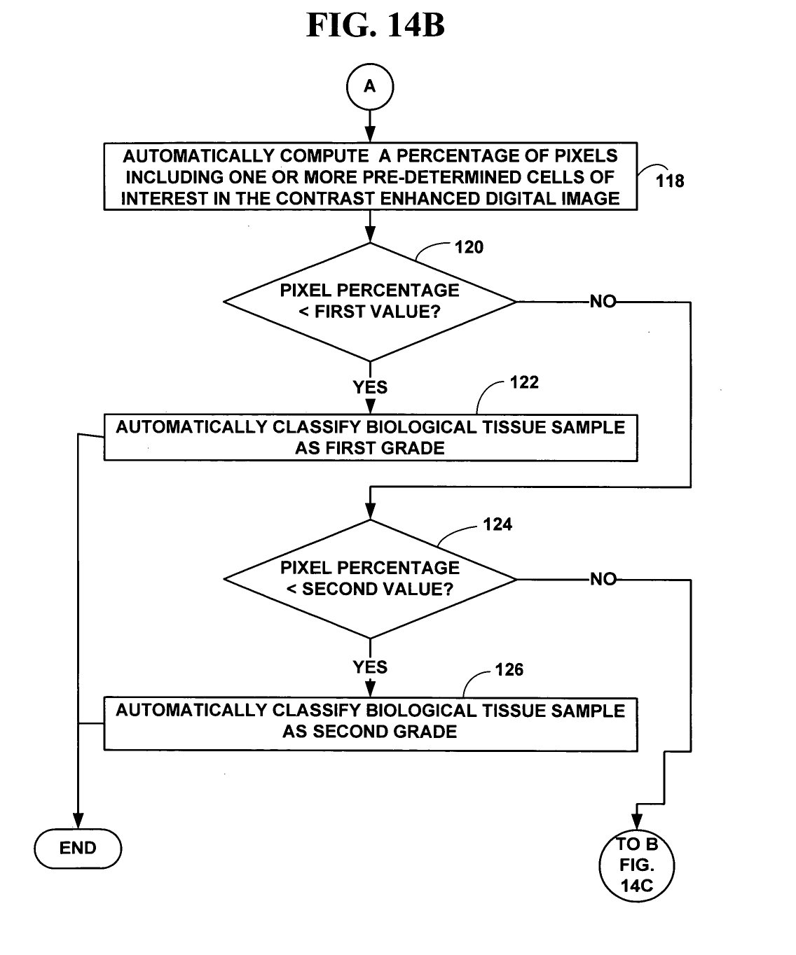

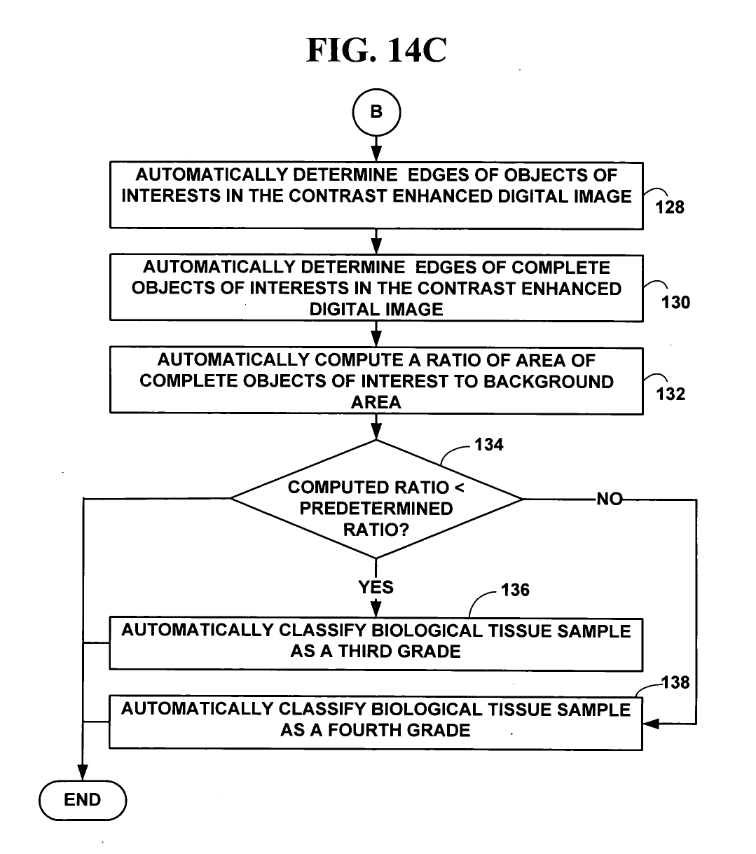

FIGS. 14A, 14B and 14C are a flow diagram illustrating an automated Method 102 for measuring morphological features in digital images. In FIG. 14A at Step 104, plural areas of interest are automatically computed for a biological tissue sample within a digital image. At Step 106, image statistics are automatically computed for the digital image. At Step 108, a contrast of the digital image is automatically enhanced using the computed image statistics. In one embodiment of the invention, the contrast is enhanced using Equation 10. However, the present invention is not limited to this embodiment and other techniques can also be used at Step 108 to practice the invention.
Enhanced pixel value=[(pixel value−X)*(mean+standard deviation/Y)]+[(X−mean)], (10)
where X and Y are predetermined constants. In one exemplary embodiment of the invention, the predetermined constants, include for example, X=128 and Y=100. However, the present invention is not limited to these constants and other constants can also be used in Equation 10.

At Step 110, a histogram 50 of grayscale values is automatically computed on the contrast enhanced digital image. At Step 112, plural grayscale intensity values are automatically determined based on a first peak 54 in the histogram 50. At Step 114, the contrast enhanced digital image is automatically segmented by thresholding the determined grayscale intensity values with grayscale values from the histogram 50. At Step 116, color correction values are automatically computed for the contrasted enhanced digital image. In one embodiment of the invention, the color a correction value is computed by computing a mean for a first color plane, computing a mean for a second color plane and then dividing the mean for the first color plane by the mean for the second color plane. In one embodiment of the invention, the first color plane is a red color plane, the second color plane is a blue color plane as was illustrated by Equation 5. However, the present invention is not limited to such an embodiment and other embodiments can also be used to practice the invention.

In FIG. 14B at Step 118, a percentage of pixels including one or more predetermined cells of interest in the digital image are automatically computed. In one embodiment of the invention, for every cell pixel, the pixel is counted if it is a stained cell pixel (e.g., brown in color if stained with IHC and if a first color component of the pixel (e.g., the Red color component if stained with IHC) is greater than (C_{f}*a second color component (e.g., the Blue color component if stained with IHC) of the pixel. Otherwise it is counted as non-stained cell pixel. A stained pixel percentage is calculated as is illustrated in Equation 11.
Stained pixel percentage=Stained cell pixels/(stained cell pixels+non stained pixels) (11)

At Step 120, a test is conducted to determine if a computed pixel percentage is less than a first value. If the computed pixel percentage is less than a first value, at Step 122 the biological tissue sample is automatically classified as a first grade. In one embodiment of the invention, the computed pixel percentage is 5% and the first grade is a HER-2/neu grade of 0+. However, the present invention is not limited to this embodiment and other pixel percentages and other grades can also be used to practice the invention.

At Step 124, a test is conducted to determine if a computed pixel percentage is less than second value. If the computed pixel percentage is less than the second value, at Step 126, the biological tissue sample is automatically classified as a second grade. In one embodiment of the invention, the computed pixel percentage is 15% and the second grade is a HER-2/neu grade of 1+. However, the present invention is not limited to this embodiment and other pixel percentages and other grades can also be used to practice the invention.

In FIG. 14C at Step 128, edges of objects of interest are automatically determined in the contrast enhanced digital image. In one embodiment of the invention, pixels on a cell membranous mesh are determined based on an intensity in the segmented image. A membrane pixel will have lower intensity value in a color plane (e.g., blue) than its two neighbors in any one of four selected directions (e.g., zero, 45, 90 and 135 degrees). However, the present invention is not limited to this embodiment and other embodiments may be used to practice the invention. At Step 130, edges of complete objects are automatically determined in the contrast enhanced digital image. In one embodiment of the invention, cells with complete membranes are determined. However, the present invention is not limited to this embodiment and other objects can also be determined to practice the invention. At Step 132, a ratio of an area of complete objects of interest to a background area is computed.

At Step 134, a test is conducted to determine whether the computed ratio is less than a predetermined ratio. If the computed ratio is less than a predetermined ratio, than at Step 136 the biological tissue sample is automatically classified as a third grade. If the computed ratio is not less than a predetermined ratio, then at Step 138 the biological tissue sample is classified as a fourth grade. In one embodiment of the invention, a ratio of mesh over the stained cell area is used to decide upon a final grade. If a ratio of mesh pixels/stained cell pixels is less than 7%, then it receives a HER-2/neu grade of 2+. Otherwise it receives a HER-2/neu grade of 3+. A predetermined ratio constant of 7% used here was derived from Equations (7) and (8). However, the present invention is not limited to this constant and other constants can also be used.

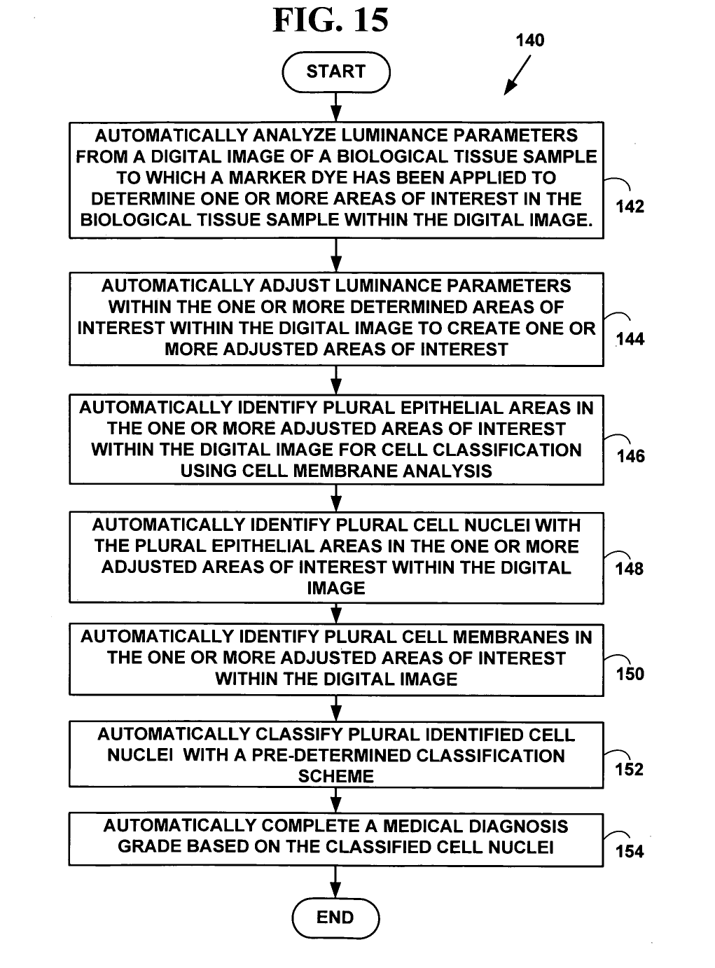

FIG. 15 is a flow diagram illustrating an automated Method 140 for biological sample analysis. At Step 142, luminance parameters from a digital image of a biological tissue sample to which a marker dye has been applied are automatically analyzed to determine one or more areas of interest in the biological tissue sample within the digital image. At Step 144, luminance parameters within the one or more determined areas of interest within the digital image are automatically adjusted to create one or more adjusted areas of interest. At Step 146, plural epithelial areas in the one or more adjusted areas of interest within the digital image are automatically identified for cell classification using cell membrane analysis. At Step 148, plural cell nuclei within the plural epithelial areas in the one or more adjusted areas of interest within the digital image are automatically identified. At Step 150, plural cell membranes in the one or more adjusted areas of interest within the digital image are automatically identified. At Step 152, plural identified cell nuclei are automatically classified with a pre-determined classification scheme. At Step 154, a medical diagnosis grade based is automatically computed based on the classified cell nuclei.

Method 140 is illustrated with one exemplary embodiment. However, the present invention is not limited to such an embodiment and other embodiments can also be used to practice the invention. In such an exemplary embodiment, the marker dye includes IHC staining of human tissue samples including plural human cells.

In such an embodiment at Step 142, luminance parameters from a digital image of a biological tissue sample to which a marker dye has been applied are automatically analyzed to determine one or more areas of interest in the biological tissue sample within the digital image. Identifying the area of interest is done by excluding the potential background which is a non tissue part. Images captured through digital devices appear to be rectangular in shape, which may or may not be the tissue shape. Tissue shape is often circular in shape. Identifying area of tissue, eliminating the non tissue part surrounding a tissue area reduces the computational effort in subsequent analysis steps. A digital image is scanned row-wise first from left to right till a pixel belonging to tissue is encountered. All pixels in a row are scanned till a tissue pixel is encountered. Pixels belonging to background or non tissue are either too dark or too bright based on their luminosity. If the background pixels are white or transparent, then their red, green and blue component values will be high, greater than a first set of pre-determined constants. If the background pixels are dark, then their red, green and blue color component values will be low, less than a second set of pre-determined constants. In other words, pixels belonging to tissue will have color component values in a range defined by minimum value of the first set of constants and a maximum value in the second set of pre-determined constants.

However, the present invention is not limited to the scanning described and other scanning techniques can also be used to practice the invention.

Table 2 illustrates exemplary values for the first and second set pre-determined constants. However, the present invention is not limited to these values and other values can be used for the first and second set of pre-determined constants. In addition all of the values in a set of pre-determined constants do not have to include the same value.

TABLE 2

First Set of Pre-Determined Constants
White background Blue Value
Constant 1.1 (Default value = 200)
White background Green Value
Constant 1.2 (Default value = 200)
White background Red Value
Constant 1.3 (Default value = 200)
Second Set of Pre-Determined Constants
Black background Blue Value
Constant 2.1 (Default value = 5)
Black background Green Value
Constant 2.2: (Default value = 5)
Black background Red Value
Constant 2.3: (Default value = 5)

The digital image is scanned from left to right. This step is repeated for three other combinations for each row of pixels and each column of pixels, namely right to left of each row, top to bottom of each column and bottom to top for each column. These steps together identify one or more areas of interest within the digital image. However, the present invention is not limited to this type of scanning and other scanning methods can also be used.

At Step 144, luminance parameters within the one or more determined areas of interest within the digital image are automatically adjusted to create one or more adjusted areas of interest. Image Enhancement: Digital images captured through an optical microscope resemble the view a human pathologist gets through optical system of a microscope. However, human pathologist is in a position to easily distinguish between nuclei, cytoplasm, red blood cells, membranous pattern and fibrin, even though there are variations in staining or variations in illumination across slide. This is because of the pathologist's experience and knowledge of the domain. Elimination of mask color and increasing the contrast between various pixels is done as a part of image enhancement.

Color correction: An image is of high color contrast if all three color levels range from minimum value (e.g., zero) to maximum value (e.g., 255). In the case of low color contrast images, this range could be as small as 50, for example from 100 to 150. If the color contrast is high, the pixels belonging to nuclei looks blue, membrane looks dark brown, cytoplasm looks moderate white and vacuoles will be of white or transparent. Color correction is required for less stained images.

Automatic adjustment done for each of the three colors, red green and blue is completed such that pixels darker than a mean of a color plane should become even more darker and pixels brighter than a mean of a color plane should become even more brighter, provided these pixel values does not exceed a maximum value. The mean of each color plane is mapped such that a resultant image mean will be some shade of white. In other words, a mask color is removed and making the mean as white as possible. Mean values for each color plane are computed using image statistics.

Equations 12, 13, and 14 are used to automatically compute modified Red, Green and Blue component values of a pixel in the digital image. However, the present invention is not limited to this embodiment and other equations can also be used to practice the invention.
Red color intensity=max(min(X, (2*Pixel Intensity−Red Mean)*BlueToRedMeanRatio), 0) (12)
Green color intensity=max(min(X, (2*Pixel Intensity−Green Mean)), 0) (13)
Blue color intensity=max(min(X, (2*Pixel Intensity−Blue Mean)*RedToBlueMeanRatio), 0) (14)
where the BlueToRedMeanRatio, and RedToBlueMeanRatio is illustrated by Equations 15 and 16.
BlueToRedMeanRatio=Blue Mean/Red Mean (15)
RedToBlueMeanRatio=Red Mean/Blue Mean (16)

Automatic modification done for each of the three colors, red green and blue is completed. In the Equation 12, a contrast in red plane pixels is increased. If the pixel has a red component value less than a mean of red plane, the term (2*Pixel Intensity−Red Mean) will be less than Pixel Intensity. If the pixel has red component value greater than the mean of red plane, the term (2*Pixel Intensity−Red Mean) will be greater than Pixel Intensity. Therefore the difference between two pixel values, one greater than mean and the other less than mean will be increased. A multiplication factor RedToBlueMeanRatio is used to normalize mean intensity of red color plane. A minimum condition used in the equation ensures that the Red component never exceeds a pre-determined constant X (e.g., 255) and maximum condition used ensures that the Red component value never becomes negative.

In Equation 13, contrast in the Green plane pixels is increased. If the pixel has Green component value less than a mean of a Green plane, the term (2*Pixel Intensity−Green Mean) will be less than Pixel Intensity. If the pixel has Green component value greater than the mean of Green plane, the term (2*Pixel Intensity−Green Mean) will be greater than Pixel Intensity. Therefore the difference between two pixel values, one greater than mean and the other less than mean will be increased. A minimum condition used in the equation ensures that the Green component never exceeds a pre-determined constant X, (e.g., 255) and a maximum condition used ensures that the Green component value never becomes negative.

In Equation 14, contrast in the Blue plane pixels is increased. If the pixel has Blue component value less than a mean of a Blue plane, the term (2*Pixel Intensity−Blue Mean) will be less than Pixel Intensity. If the pixel has Blue component value greater than the mean of Blue plane, the term (2*Pixel Intensity−Blue Mean) will be greater than Pixel Intensity. Therefore the difference between two pixel values, one greater than mean and the other less than mean will be increased. A minimum condition used in the equation ensures that the Blue component never exceeds a predetermined constant X, (e.g., 255) and a maximum condition used ensures that the Blue component value never becomes negative.

At Step 146, plural epithelial areas in the one or more adjusted areas of interest within the digital image are automatically identified for cell classification using cell membrane analysis. A Gaussian kernel is used for weighted averaging of pixels in a small window centered around a given pixel. Keeping a window size equal to the width of two typical epithelial cells, a differentiation can be determined between the densely packed epithelial area and stromal area. Weighted averages using a Gaussian kernel typically are very high in the stromal area.

In one embodiment of the invention, the Gaussian kernel uses the constants listed in Table 3. However, the present invention is not limited to these values and other values can be used for the constants used in the Gaussian kernel.

TABLE 3

Gaussian Sigma used to represent cell size in epithelial area
Constant 3.1 (Default value = 5)
Intensity Value for Epithelial Area
Constant 3.2: (Default value = 128)

In one embodiment of the invention, a Gaussian kernel of sigma three is used as is illustrated in Equation 17. However, the present invention is not limited to this embodiment another other Gaussian kernels can also be used to practice the invention.
Gaussian kernel f(x)=power(e−constantG*x*x/(Sigma*Sigma))/(Sigma*sqrt(2*pi)) (17)
Where e=“2.71828 . . . ” and constantG=0.5. However, the present invention is not limited to a constantG of 0.5 and other values can be used to practice the invention. A Gaussian kernel is used for convolution with a modified image as is illustrated in Equation 18.

$$G = {\sum }_{x = - ( kernelsize / 2 )}^{x = kernselsize / 2} f ( x ) * Ix , \qquad ( 18 )$$

where “G” is a Gaussian value at a color position, “kernel size”=1+2*ceiling(2.5*Sigma) and “Ix” is a pixel value at x. Pixels that are on a curve of symmetry of epithelial cell or epithelial area are marked. Typically there will be two curves of symmetry, one parallel to x-axis and the other parallel to y-axis. Pixels belonging to region of interest are selected based on the intensity. Pixels with intensity value less than (Mean+Standard Deviation) of the image are selected as pixels belonging to region of interest.

At Step 148, plural cell nuclei with the plurality epithelial areas in the one or more adjusted areas of interest within the digital image are automatically identified. In one embodiment of the invention, in the epithelial area, individual nucleus is identified such that an extent of stained membrane around each nucleus is estimated. The Gaussian kernel concept described above in Equation 18 is also used for nuclei identification, but with a different kernel size. Since the nucleus is stained blue color with haematoxylin staining, the blue color plane of the image is used for applying a Gaussian blur. A given pixel is identified as pixel in nucleus if it satisfies the conditions and constants listed in Table 4. However, the present invention is not limited to these values and other values can be used for the conditions and constants

TABLE 4

```
Gaussian Sigma for Nuclei Identification
Constant 4.1: (Default value = 2)
Mask for nuclei identification
Constant 4.2: (Default value = 30)
Nuclei brightness
Constant 4.3: (Default value = 300)
  Pixel intensity < nuclei brightness
        Pixel is in epithelial area
  At least half of ‘n’ neighboring pixels in top, bottom,
  left and right direction have intensity more
  than the given pixel.
```

After detecting potential pixels that could be on a nucleus, a connected set of such pixels is labeled. A size of each nucleus or a number of pixels in any connected set of pixels is checked. If this size is less than a predetermined value, the set of connected pixels do not belong to a nucleus and hence removed. A predetermined value of the number of pixels is (Constant 4.2/2).

At Step 150, plural cell membranes in the one or more adjusted areas of interest within the digital image are automatically identified. In one embodiment of the invention, with IHC staining, cell membranes are brown in color and counterstained with haematoxylin, nuclei are blue in color. However variations in staining and the color of the lamp used in optical microscope might create a color background in the image. This is often called a color “mask.” Presence of a color mask might change the relation between blue and red components of a pixel.

In one embodiment of the invention, the cell nucleus and cell membranes are identified using the constants listed in Table 5. However, the present invention is not limited to these values and other values can be used for the constants to identify cell nucleus and cell membranes.

TABLE 5

Minimum Blue color in Membrane, indicating the minimum
intensity of counter-stain
Constant 5.1: (Default value = 0)
Maximum Blue color in Membrane, indicating the maximum
intensity of counter-stain
Constant 5.2: (Default value = 200)
Maximum standard deviation for uniform region
Constant 6.1 (Default value = 5)
Percentage for identifying maximum intensity of each color
Constant 6.2: (Default value = 99)
Radius of cell
Constant 6.3: (Default value = 30)

In one embodiment of the invention, color mask removal is a step carried out before membrane identification. A presence of a color mask can be detected by measuring the standard deviation in all the three color planes of image. A standard deviation will be very low or there will be uniform illumination if there is a mask. Mean and standard deviation of a histogram of each color plane is computed using standard formulae given in Equation 19.
Standard Deviation=(1/Image size)*Sum(Mean−Pixel intensity)/2 (19)

A histogram of each color plane is computed using a counting frequency of occurrence of each color level in the range. Once it is detected that there is a mask in the image, its effect is nullified by stretching histograms of the three-color planes, red, green and blue independently. An amount by which a color component of a pixel gets modified is dependent on a maximum value of that particular color component in the image and the ratio of the current pixel value to maximum pixel value. Maximum pixel intensity in each color plane is computed using a cumulative histogram. If the cumulative histogram exceeds a predetermined constant (e.g., Constant 6.2) then a maximum pixel intensity is reached. Pixels with higher intensity belong to background or mask.

A pixel in an epithelial area is considered to be on a cell membrane if its blue component satisfies the following conditions illustrated in Table 6. However, the present invention is not limited to these values and other values can be used for the conditions and constants.

TABLE 6
Blue component is greater than a predetermined value (Constant 5.1)
Blue component is less than a pre determined value (Constant 5.2)
Blue component is less than the Red component
Blue component is less than blue component of all its eight neighbors.

Those pixels that satisfy the above four conditions are identified as cell membrane pixels and are marked with red color, by setting red component of pixel to a predetermined value (e.g., 255).

At Step 152, plural identified cell nuclei are automatically classified with a pre-determined classification scheme. In one embodiment of the invention, identified need to be classified into four different categories based on the extent of a stained cell membrane ring around it. An extent of membrane ring varies from zero degrees in the case of no ring to 360 degrees in the case of full rings. Radial lines are drawn through a center of an identified nucleus in all 360 degrees and it is determined if there exists a membrane pixel on this radial line. If a length of a radial line exceeds the radius of a typical nucleus no more membrane pixels are looked for. In the case of full ring membranes, the number of membrane pixels detected by these radial lines should be 360. A ratio of a number of membrane pixels around a nucleus over 360 gives an extent of a membrane ring or a membrane percentage.

At Step 154, a medical diagnosis grade based is automatically computed based on the classified cell nuclei. In one embodiment of the invention, two different but interrelated measurements are used to get an accurate quantitation and thus a medical diagnosis. Two different but inter related measurements are carried out on the cells detected and an extent of stained cell membrane around these cells. In a first measure, distribution of cells into HER-2/neu grades 0+, 1+, 2+ and 3+ is carried out. In a second measure, appropriation of these cell categories in arriving at a score is given. In both measurements, a user has flexibility in setting limits. That is, a user can separately set limits for extent of cell membranes that decide cells into 0+, 1+, 2+ and 3+ grades.

Limits used to decide score based on the percentage of cells belonging to HER-2/neu grades 0+, 1+, 2+ and 3+ are illustrated in Table 7. However, the present invention is not limited to these values and other values can be used for the constants.

TABLE 7
Stained Membrane percentage for identifying 0+ cells Constant 6.1:
(Default value = 10)
Stained Membrane percentage for identifying 1+ cells
Constant 6.2: (Default value = 40)
Stained Membrane percentage for identifying 2+ cells
Constant 6.3: (Default value = 80)
Minimum percentage of 3+ cells for giving a score of 3 to a given sample
Constant 7.1: (Default value = 30)
Minimum percentage of 2+ cells for giving a score of 3 to a given sample
Constant 7.2: (Default value = 80)
Minimum percentage of 3+ cells for giving a score of 2 to a given sample
Constant 7.3: (Default value = 10)
Minimum percentage of 2+ cells for giving a score of 2 to a given sample
Constant 7.4: (Default value = 30)
Minimum percentage of 2+ cells for giving a score of 1 to a given sample
Constant 7.5: (Default value = 10)
Minimum percentage of 1+ cells for giving a score of 1 to a given sample
Constant 7.6: (Default value = 5)

Using Method 140 cells of interest are detected automatic based on the characteristics of nucleus, membrane and cytoplasm. In most other methods known in the art, user has to mark region of interest using some kind of interactive tools. Automated detection of nucleus, membrane and staining intensities are carried out using morphological properties of cells, which is similar to the way human pathologists analyze tissue samples. Other methods known in the art are based on optical intensity distribution in the area of interest. These methods typically do not use morphological properties of cells. The method provides a percentage distribution of membrane staining per nucleus and using a Gaussian blur is typically rugged and reliable.

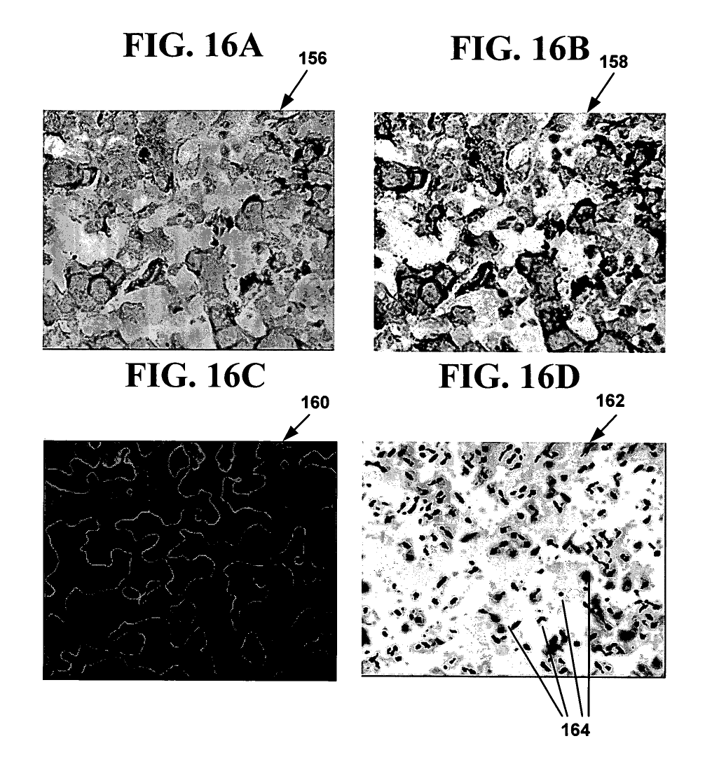

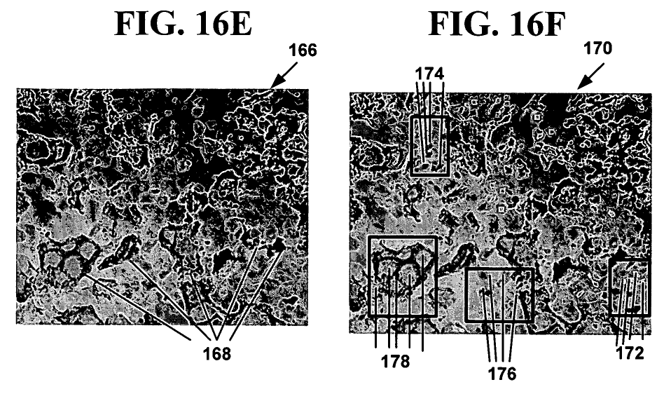

FIG. 16. illustrates a sample digital image containing cells with varying HER-2/-neu amplification. This image has cells with a clear membranous pattern (e.g., 3+) and epithelial cells without any membrane (e.g., 0+).

FIG. 16A is a block diagram 156 illustrating an original sample digital image. FIG. 16B is a block diagram 158 illustrating the digital image of FIG. 16A automatically adjusted at Step 144. FIG. 16C is a block diagram 160 illustrating plural epithelial areas of the original image of FIG. 16A automatically identified at Step 146. FIG. 16D is a block diagram 162 illustrating plural nuclei 164 of the original image of FIG. 16A automatically identified at Step 148. FIG. 16E is a block diagram 166 illustrating plural cell membranes 168 automatically identified in the original image of FIG. 16A at Step 150. FIG. 16F is a block diagram 170 illustrating an automatic cell classification based on plural membranous patterns, nuclei and membranes at Step 152.

Block diagram 170 illustrates identification of plural exemplary (but not all) clusters of 0+ cells 172, 1+ cells 174, 2+ cells 176 and 3+ cells 178 automatically classified at Step 152. A medical diagnosis is automatically completed at Step 154 based on the percentages of 0+, 1+, 2+ and 3+ cells classified at Step 152.

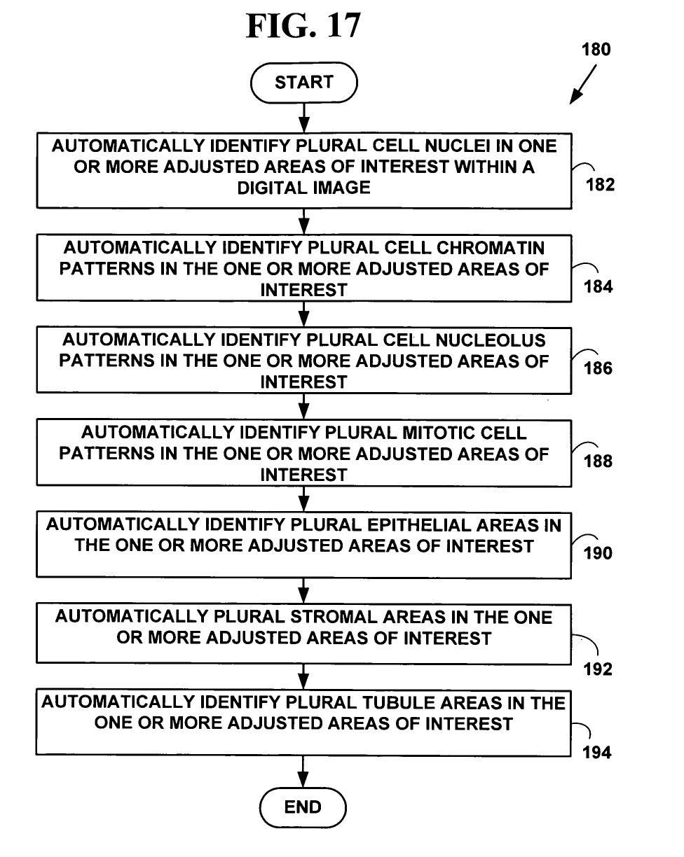

FIGS. 17-26 illustrate details of methods that are used to practice one or more steps of Methods 20, 38, 94, 102 and 140. However, Methods 20, 38, 94, 102 and 140 are not limited to using the methods described in FIGS. 17-26 and other methods can be used to practice the various steps of these methods to practice the invention.

FIG. 17 is a flow diagram illustrating a Method 180 for identification of individual morphological components from one or more adjusted areas of interest within the digital image of a biological tissue sample to which a marker dye has been applied. At Step 182, plural cell nuclei from the one or more adjusted areas of interest within the digital image are automatically identified. At Step 184, plural cell chromatin patterns from the one or more adjusted areas of interest within the digital image are automatically identified. At Step 186, plural cell nucleolus patterns from the one or more adjusted areas of interest within the digital image are automatically identified. At Step 188, plural mitotic cells from the one or more adjusted areas of interest within the digital image are automatically identified. At Step 190, plural epithelial areas from the one or more adjusted areas of interest within the digital image are automatically identified. At Step 192, plural stromal areas from the one or more adjusted areas of interest within the digital image are automatically identified. At Step 194, plural tubule areas from the one or more adjusted areas of interest within the digital image are automatically identified.

In one embodiment, Method 180 is used, but is not limited to, for example, at Step 24 of Method 20, Step 34 of Method 28 and Step 96 of Method 94. However, the invention is not limited to using Method 180 at these steps and other steps can be used to practice the invention.

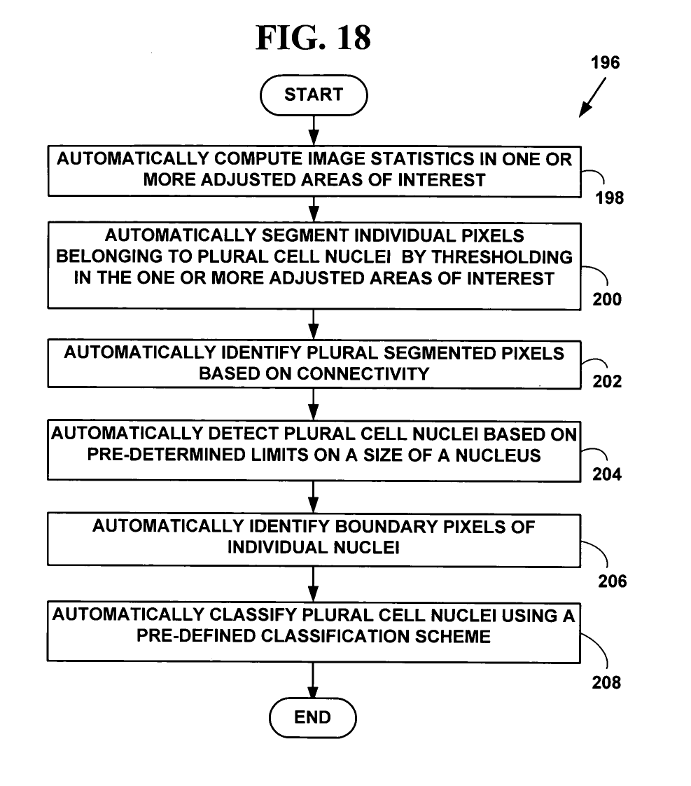

FIG. 18 is a flow diagram illustrating a Method 196 for identification of cell nuclei from one or more adjusted areas of interest within the digital image of a biological tissue sample to which a marker dye has been applied. At Step 198, image statistics (e.g., mean and standard deviation in each of the red, green and blue color planes) in the one or more adjusted areas of interest are automatically computed. At Step 200, individual pixels belonging to plural cell nuclei are automatically segmented by thresholding in the one or more adjusted areas of interest. At Step 202, plural segmented pixels are automatically identified based on connectivity. At Step 204, one or more cell nuclei are automatically deleted from the adjusted area of interest based on pre-determined limits in a size of a nucleus (e.g., number of connected pixels after segmentation). At Step 206, boundary pixels of individual nuclei are automatically identified. At Step 208, plural cell nuclei are automatically classified using a pre-defined classification scheme.

In one embodiment, Method 196 is used, but is not limited to, for example, at Step 114 of Method 102 and Step 148 of Method 140. However, the invention is not limited to using Method 196 at these steps and other steps can be used to practice the invention.

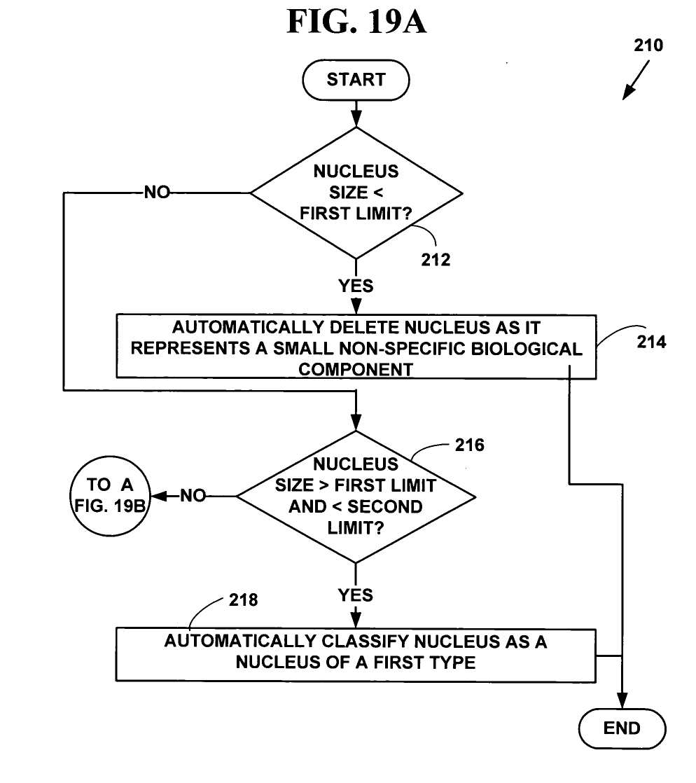

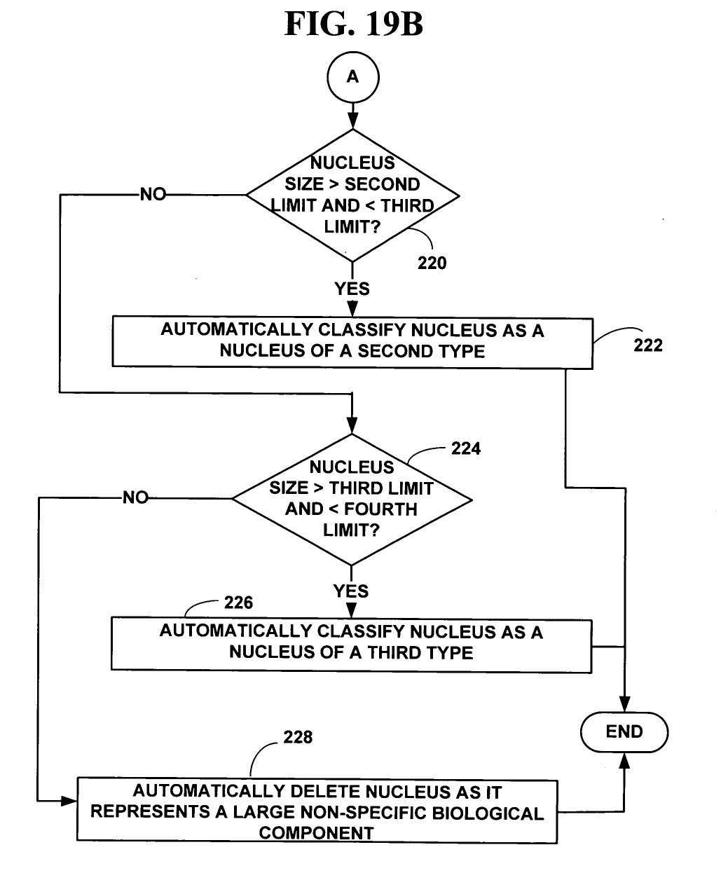

FIGS. 19A and 19B are a flow diagram illustrating a Method 210 for classification of cell nuclei according to a pre-defined classification scheme. Method 210 uses number of connected pixels in a segmented nucleus for classifying nucleus into three types. However, the present invention is not limited to such an embodiment and other embodiments can also be used to classify cell nuclei into more or fewer types.

In FIG. 19A, at Step 212 a test is conducted to determine if an identified nucleus size in pixels less than a first pre-defined limit. If so, at Step 214, the nucleus is filtered or deleted as these size nuclei represent small non-specific biological components (e.g., dust particles, lymph cells). At Step 216, a test is conducted to determine if the identified nucleus size in pixels is greater than the first pre-defined limit and less than a second pre-defined limit. If so, at step 218, the identified nucleus is classified as a first nucleus type.

In FIG. 19B at Step 220, a test is conducted to determine if the identified nucleus size in pixels is greater than the second pre-defined limit and less than a third pre-defined limit. Is so, at Step 222, the identified nucleus is classified as a second type. At Step 224, a test is conducted to determine if the identified nucleus size in pixels is greater than a third pre-defined limit and less than a fourth pre-defined limit. If so, at Step 226, the identified nucleus is classified as a third type. If the identified nucleus is larger than the fourth pre-defined limit, at Step 228 such identified nuclei larger than the fourth pre-defined limit are deleted as a large non-specific biological component (e.g., artifacts, blood vessels).

In one embodiment, Method 210 is used, but is not limited to, for example, is not limited to using Method 210 at these steps and other steps can be used to practice the invention.

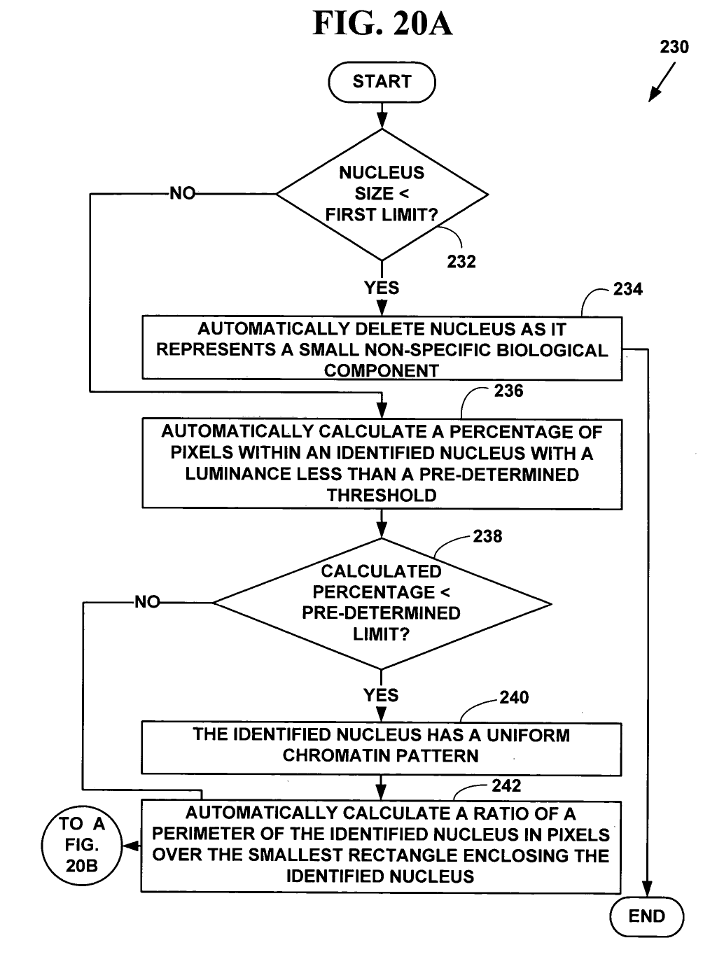

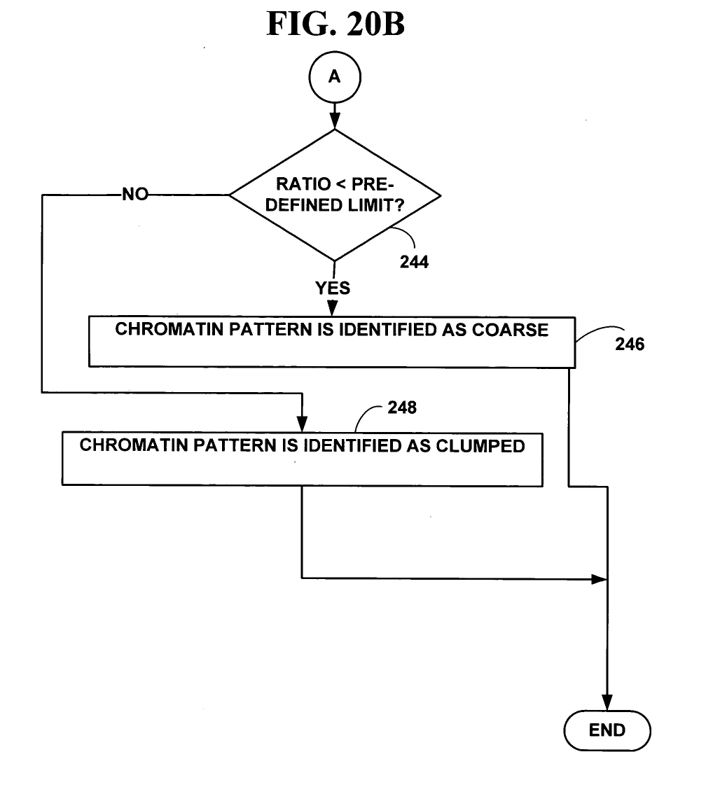

FIGS. 20A and 20B are a flow diagram illustrating a Method 230 for classification of cell chromatin pattern according to a pre-defined classification scheme. Method 230 uses number of connected pixels in a segmented nucleus and the geometry of the connected pixels for classifying chromatin pattern into three types. In FIG. 20A at Step 232, a test is conducted to determine if an identified nucleus size in pixels is less than a first pre-defined limit. If so, at Step 234 the nucleus is filtered or deleted as a small nucleus represents non-specific biological components (e.g., dust particles, lymph cells). At Step 236, a percentage of pixels within an identified nucleus with a luminance less than a pre-determined threshold is calculated. At Step 238, a test is conducted to determine if this calculated percentage is less than a pre-defined level. If so, at Step 240, the identified nucleus has a “uniform” chromatin pattern. At Step 242, a ratio of a perimeter of the identified nucleus in pixels over the smallest rectangle enclosing the identified nucleus is calculated.

In FIG. 20B at Step 244, a test is conducted to determine if the calculated ratio is less than a pre-defined limit. If so, at Step 246, the chromatin pattern in the identified nucleus is classified as “coarse.” If not at Step 246, the chromatin pattern in identified nucleus is classified as “clumped.”

In one embodiment, Method 230 is used, but is not limited to, for example, at Step 24 of Method 20, Step 34 of Method 28 and Step 96 of Method 94. However, the invention is not limited to using Method 230 at these steps and other steps can be used to practice the invention.

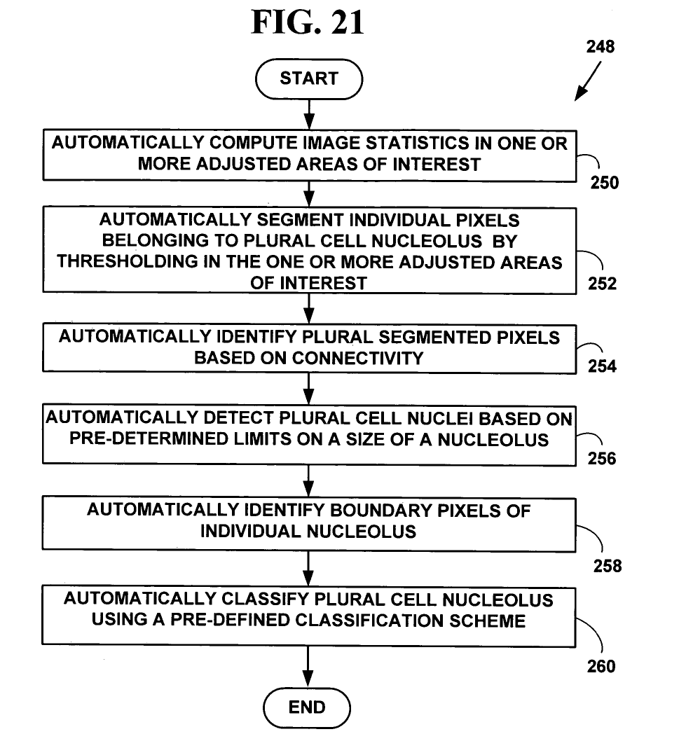

FIG. 21 is a flow diagram illustrating a Method 248 for identification of cell nucleolus from one or more adjusted areas of interest within the digital image of a biological tissue sample to which a marker dye has been applied. At Step 250, image statistics (e.g., mean and standard deviation in each of the red, green and blue color planes) in the area of interest are automatically calcuated. At Step 252, individual pixels belonging to cell nucleolus are automatically segmented by thresholding contrast modified digital image in the area of interest. At Step 254, plural segmented pixels are automatically identified based on connectivity. At Step 256, plural cell nucleolus are automatically deleted from area of interest within the digital image based on pre-determined limits for a size of nucleolus (e.g., number of connected pixels after segmentation). At Step 258, boundary pixels of individual nucleolus are automatically identified. At Step 260, plural nucleolus are automatically classified using a pre-defined classification scheme.

In one embodiment, Method 248 is used, but is not limited to, for example, at Step 96 of Method 94, Step 114 of Method 102 and Step 148 of Method 140. However, the invention is not limited to using Method 248 at these steps and other steps can be used to practice the invention.

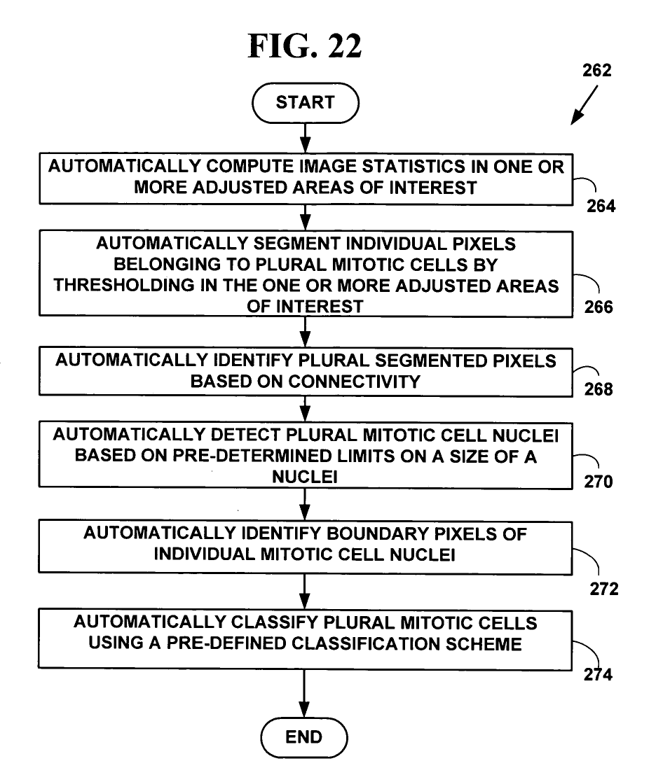

FIG. 22 is a flow diagram illustrating a Method 262 for identification of mitotic cells from one or more adjusted areas of interest within the digital image of a biological tissue sample to which a marker dye has been applied. At Step 264, image statistics (e.g., mean and standard deviation in each of the red, green and blue color planes) in the area of interest are automatically calculated. At Step 266, individual pixels belonging to mitotic cells are automatically segmented by thresholding contrast modified digital image in the area of interest. At Step 268, plural segmented pixels are automatically identified based on connectivity. At Step 270, one or more mitotic cell nuclei are automatically deleted from area of interest within the digital image of a biological tissue based on pre-determined limits in the size of nucleus (e.g., number of connected pixels after segmentation). At Step 272, boundary pixels of individual mitotic cell nuclei are automatically identified. At Step 274, mitotic cell pattern are automatically classified using a pre-defined classification scheme.

In one embodiment, Method 262 is used, but is not limited to, for example, at Step 96 of Method 94, Step 114 of Method 102 and Step 148 of Method 140. However, the invention is not limited to using Method 262 at these steps and other steps can be used to practice the invention.

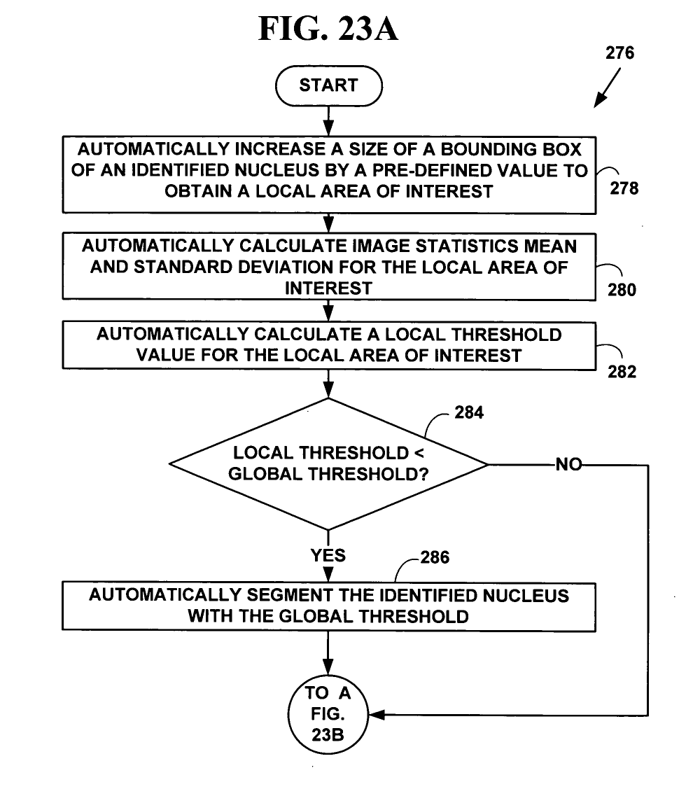

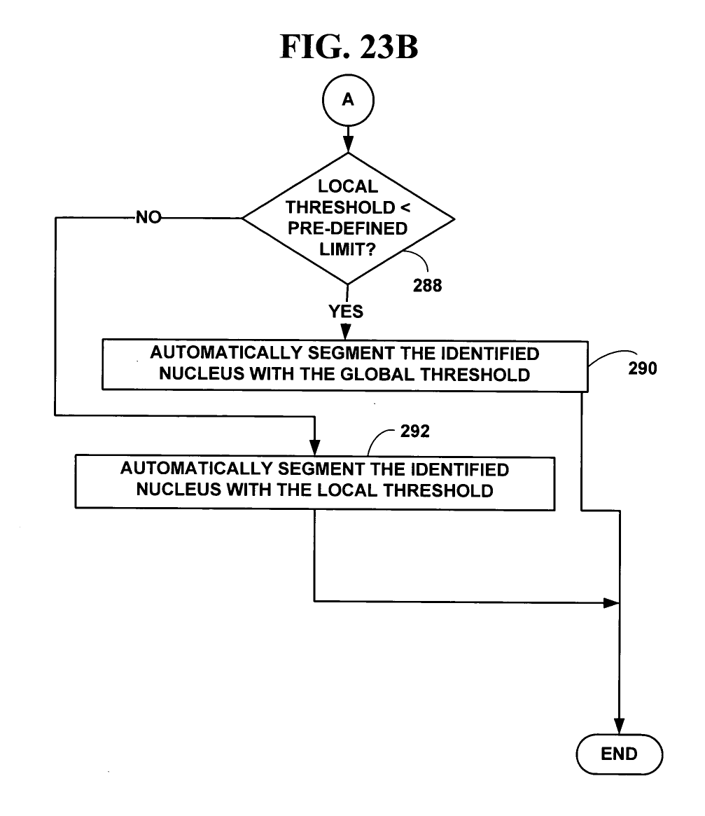

FIGS. 23A and 23B are a flow diagram illustrating a Method 276 for detecting boundary pixels of an identified nucleus. In FIG. 23A at Step 278, a size of a bounding box of an identified nucleus is automatically increased by a pre-defined value to obtain a local area of interest. At Step 280, image statistics mean and standard deviation are automatically calculated for the local area of interest. At Step 282, a local threshold value for the local area of interest is automatically calculated. At Step 284, a test is conducted to determine if the local threshold value is less than a global image threshold value. If so, Step 286, the identified nucleus is automatically segmented with the global threshold.

In FIG. 23B at Step 288, a test is conducted to determine if the local threshold is greater than a pre-defined limit. If so, at Step 290 the identified nucleus is automatically segmented with the global threshold. If not, at Step 292, the identified nucleus is automatically segmented with the local threshold.

In one embodiment, Method 276 is used, but is not limited to, for example, at Step 96 of Method 94, Step 114 of Method 102 and Step 148 of Method 140. However, the invention is not limited to using Method 276 at these steps and other steps can be used to practice the invention.

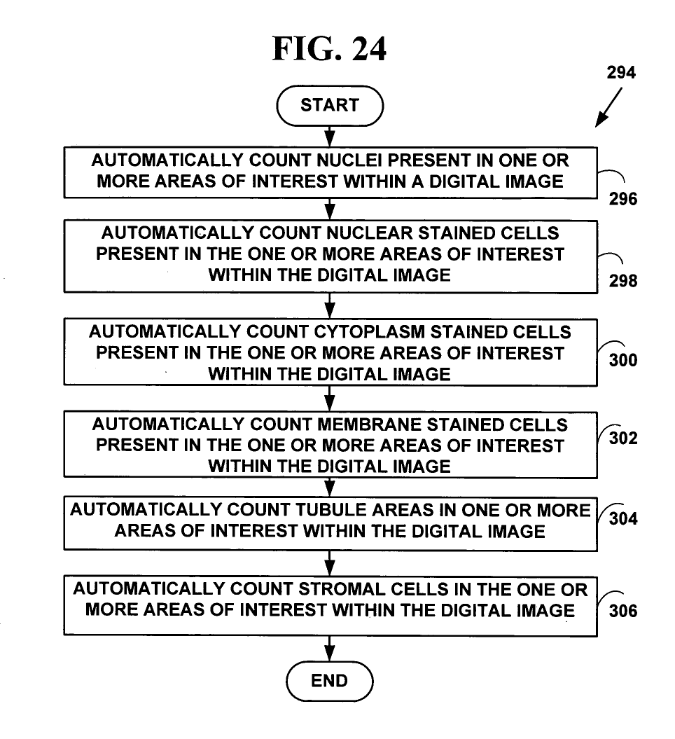

FIG. 24 is a flow diagram illustrating a Method 294 for counting individual biological components from one or more adjusted areas of interest within the digital image of a biological tissue sample to which a marker dye has been applied. At Step 296, nuclei present in the one or more areas of interest within the digital image are automatically counted. At Step 298, nuclear stained cells present in one or more areas of interest within the digital image are automatically counted. At Step 300, cytoplasm stained cells present in one or more areas of interest within the digital image are automatically counted. At Step 302, membrane stained cells present in the one or more areas of interest within the digital image are automatically counted. At Step 304, tubule areas in the one or more areas of interest within the digital image are automatically counted. At Step 306, stromal cells in the one or more areas of interest within the digital image are automatically counted.

In one embodiment, Method 294 is used, but is not limited to, for example, at and Step 96 of Method 94, Step 114 of Method 102 and Step 148 of Method 140. However, the invention is not limited to using Method 294 at these steps and other steps can be used to practice the invention.

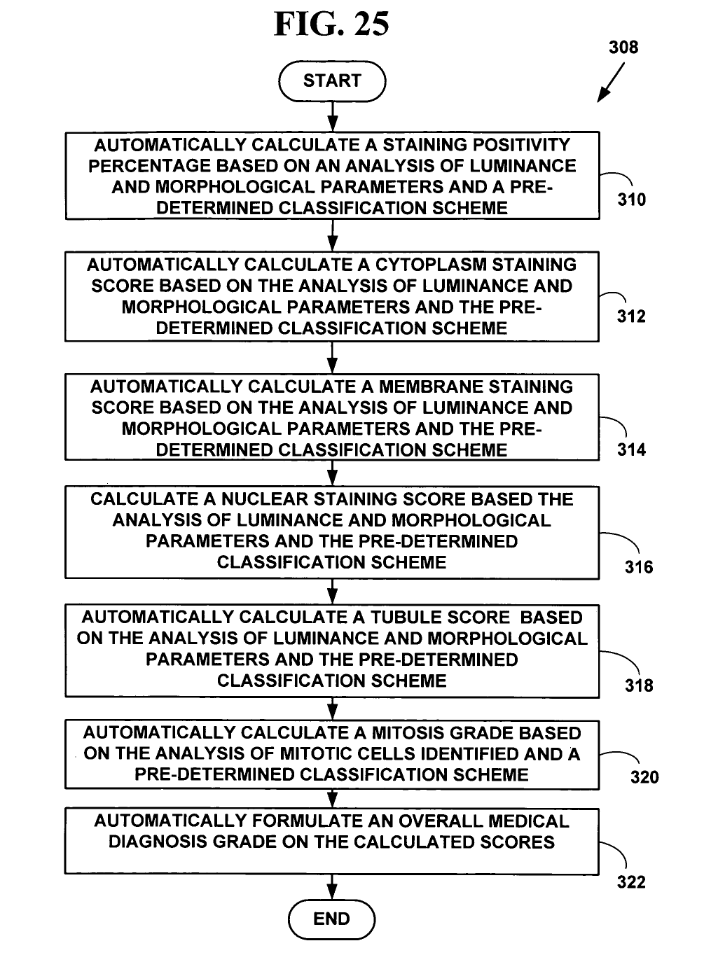

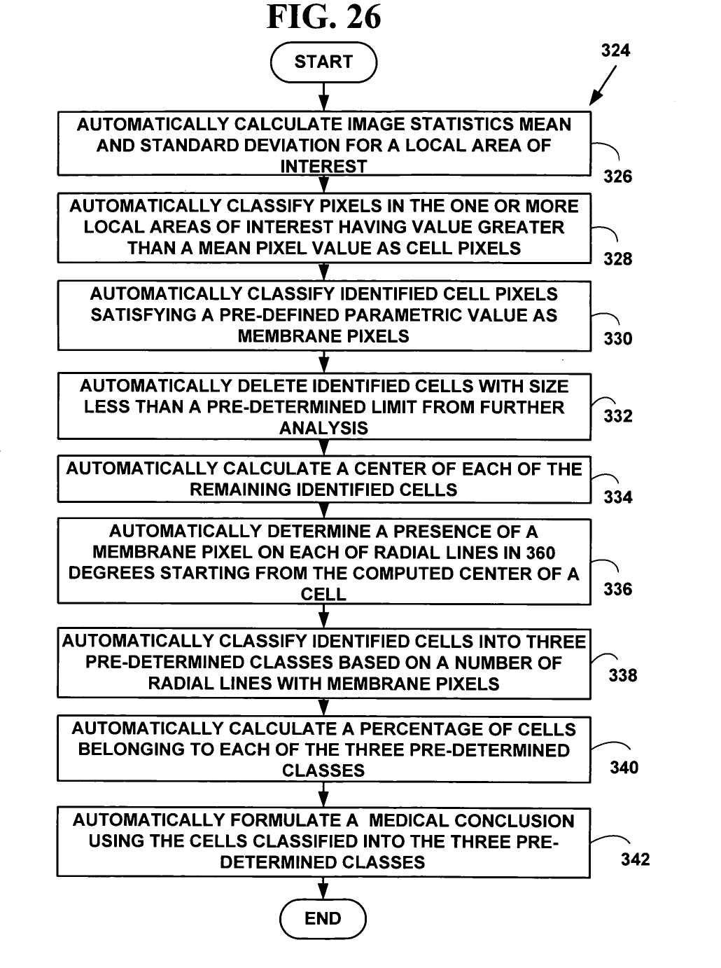

FIG. 25 is a flow diagram illustrating a Method 308 for quantification of individual biological components and tissue sample based on one or more adjusted areas of interest within the digital image of a biological tissue sample to which a marker dye has been applied. At Step 310, a staining positivity percentage is automatically calculated based on an analysis of luminance and morphological parameters and a pre-determined classification scheme. At Step 312, a cytoplasm staining score is automatically calculated based on the analysis of luminance and morphological parameters and the pre-determined classification scheme. At Step 314, a membrane staining score is automatically calculated based on the analysis of luminance and morphological parameters and the pre-determined classification scheme. At Step 316, a nuclear staining score is automatically calculated based the analysis of luminance and morphological parameters and the pre-determined classification scheme. At Step 318, a tubule score is automatically calculated based on the analysis of luminance and morphological parameters and the pre-determined classification scheme. At Step 320, a mitosis grade is automatically calculated based on the analysis of mitotic cells identified and a pre-determined classification scheme. At Step 322, an overall medical diagnosis grade is automatically calculated using the scores automatically calculated at Steps 310-322.

In one embodiment, Method 308 is used, but is not limited to, for example, at Step 26 of Method 20, Step 36 of Method 28, Step 100 of Method 94 and Step 154 of Method 140. However, the invention is not limited to using Method 308 at these steps and other steps can be used to practice the invention.

FIG. 26 is a flow diagram illustrating a Method 324 for quantifying membrane stain values in identified cells in areas of interest within the digital image of a biological tissue sample to which a marker dye has been applied. At Step 326, image statistics mean and standard deviation are automatically calculated for a local area of interest. At Step 328, pixels in the one or more local areas of interest having value greater than a mean pixel value are automatically classified as cell pixels. At Step 330, identified cell pixels satisfying a pre-defined parametric value are automatically classified as membrane pixels. At step 332, identified cells with size less than a pre-determined limit are automatically deleted from further analysis. At Step 334, a center of each of the remaining identified cells is automatically calculated. At Step 336, a presence of a membrane pixel on each of radial lines in 360 degrees starting from the computed center of a cell is automatically determined. At Step 338, identified cells are automatically classified into three pre-determined classes based on a number of radial lines with membrane pixels. At Step 340, a percentage of cells belonging to each of the three pre-determined classes is automatically calculated. At Step 342, a medical conclusion is formulated using the cells classified into the three pre-determined classes.

In one embodiment, Method 324 is used, but is not limited to, for example, at Step 26 of Method 20, Step 36 of Method 28, Step 100 of Method 94 and Step 154 of Method 140. However, the invention is not limited to using Method 324 at these steps and other steps can be used to practice the invention.

Various cell component factors are considered to practice Method 324. The cell component factors, include, but are not limited to, nuclei brightness, an elongation ratio, minimum nucleus size. These factors allow for a better medical diagnosis or medical prognosis or life science or biotechnology experiment conclusion to be reached. However, the present invention is not limited to factors and other factors can also be used to practice the invention.

Nuclei brightness is an indication of the luminance parameter of nuclei. Increasing a pre-determined threshold of nuclei brightness allows additional nuclei to be considered in the digital image analysis. In addition, decreasing the pre-determined threshold of nuclei brightness eliminates excessive nuclei from consideration in the digital image analysis.

An elongation ratio indicates the ratio of a major axis over a minor axis of a nucleus. This ratio approaches unity for circular shape nuclei, and this ratio will be large for non-circular nuclei (e.g., stromal cells). Decreasing the pre-determined threshold of elongation ratio eliminates elliptical shape nuclei in the digital image analysis. Increasing the pre-determined threshold of elongation ratio includes elliptical nuclei into the digital image analysis.

Minimum nucleus size indicates a smallest size object identified as nucleus. Decreasing the pre-determined threshold of a minimum nucleus size will include smaller nucleus as well as other cell components such a few lymph cells and large dust particles as nuclei in the digital image analysis. Increasing the pre-determined threshold eliminates some genuine nuclei as well as large dist particles, lymph cells from digital image analysis.

In one embodiment of Method 324, modification to the membrane staining measurements is done. Membrane staining intensity, the extent of stained membrane around a nucleus and the percentage of membrane stained cells within area of interest of digital image of a biological tissue sample is used in arriving at life science and biotechnology experiment conclusion.

In one embodiment of the invention, a medical diagnosis at Step 342 is automatically formulated based on HER-2/neu over-expression scoring including, but not limited to: “1+,” for faint, equivocal, and incomplete membranous staining; “2+,” for unequivocal, complete membranous patterns, with moderate intensity; and “3+,” for strong, membranous patterns.

In one embodiment of the invention for HER-2/neu grading, a cell ring is a complete brown cell ring and a number of stained pixels used is a number of stained brown pixels. In such an embodiment, the cells from the biological tissue sample have been stained with IHC staining and counter stained with haematoxylin. However, the present invention is not limited to brown colored cells and if other stains are used, then cells stained with other colors are used to practice the invention.

In one embodiment of the invention for HER-2/neu grading, modification to membrane staining measurements is done based on the extent of membranous ring structure around a nucleus. Nucleus and cytoplasm of a cell are enclosed within a membrane. However, in tissue samples having cross sections of nuclei, membranes appear to be a ring. Staining intensity of membranes and the extent they are stained is used in digital image analysis to automatically formulate a medical diagnosis or medical prognosis. An extent of membrane staining is classified as complete ring if 360 degrees of the membrane ring is stained, as partial ring if less than 360 degrees but more than a pre-determined threshold is stained.

Complete membrane ring stained cells are those cells with stained membrane ring greater than pre-determined threshold for complete ring membrane. Decreasing this threshold will include nuclei with less than 360 degrees stained membrane ring. It is necessary to reduce this threshold if the staining intensity is low or the device used to cut tissue sample is not sharp. Sectioning a biological sample with blunt devices might result in fragmented segments of membrane rings. Increasing the threshold to its maximum ensures that only nucleus with 360 degrees membrane ring is identified as complete ring cell. In the case of over stained samples, it is required to identify complete ring stained cells to arrive at accurate medical diagnosis or prognosis or life science and biotechnology experiment conclusion

Partial membrane ring stained cells are those cells with stained membrane ring but less than 360 degrees or complete ring. Decreasing a pre-determined partial ring threshold value would include more number of nuclei in image analysis and medical diagnosis or medical prognosis or life science and biotechnology experiment conclusion. It is often necessary to reduce this threshold if the staining intensity is low. Increasing the threshold to its maximum ensures that only nucleus with 360 degrees membrane ring is identified as partial ring cell. In the case of over stained samples, it is required to identify partial ring stained cells to arrive at accurate medical diagnosis or medical prognosis or life science and biotechnology experiment conclusion.

Modification to the membrane staining score is done based on a percentage of cells with complete stained membrane ring, percentage of cells with partial stained membrane ring, thickness of the membrane, and staining intensity. Membrane staining score 0+, 1+, 2+ or 3+ is given using percentage of cells with complete stained membrane ring, percentage of cells with partial stained membrane ring, thickness of the membrane, and staining intensity.

A membrane staining score for 3+ grading is given if a percentage of cells with complete stained membrane ring are more than pre-determined threshold. Decreasing the percentage of cells with complete stained membrane ring threshold will include nuclei with less than 360 degrees stained membrane ring for assigning 3+ score to a tissue sample. It is necessary to reduce this threshold if the staining intensity is low or the device used to cut tissue sample is not sharp. Sectioning a biological sample with blunt devices might result in fragmented segments of membrane rings. Increasing the percentage of cells with complete stained membrane ring threshold ensures that only nucleus with 360 degrees membrane ring is identified as complete ring cell. In the case of over stained samples, it is required to identify complete ring stained cells to arrive at accurate medical diagnosis or medical prognosis or life science and biotechnology experiment conclusion.

Membrane staining score 3+ is given if an average membrane thickness is more than a pre-determined threshold. Decreasing the threshold will include nuclei with less than 2 pixels thick membrane ring for assigning 3+ score to a tissue sample. It is necessary to reduce this threshold if the staining intensity is low or the device used to cut tissue sample is not sharp. Sectioning a biological sample with blunt devices might result in fragmented segments of membrane rings. Increasing the threshold ensures that only nuclei with thick membrane are identified for 3+ score. In the case of over stained samples, it is required to increase membrane thickness threshold to arrive at accurate medical diagnosis or medical prognosis or life science and biotechnology experiment conclusion.

Membrane staining score 2+ is given if the percentage of cells with complete stained membrane ring are more than pre-determined threshold. Decreasing the percentage of cells with complete stained membrane ring threshold will include nuclei with less than 360 degrees stained membrane ring for assigning 2+ score to a tissue sample. It is necessary to reduce this threshold if the staining intensity is low or the device used to cut tissue sample is not sharp. Sectioning a biological sample with blunt devices might result in fragmented segments of membrane rings. Increasing the percentage of cells with complete stained membrane ring threshold ensures that only nucleus with 360 degrees membrane ring is identified as complete ring cell. In the case of over stained samples, it is required to identify complete ring stained cells to arrive at accurate medical diagnosis or life science and biotechnology experiment conclusion.

A membrane staining score 2+ is given if the if the sum of percentages of cells with complete stained membrane ring and partial stained membrane ring is more than pre-determined threshold and staining intensity is more than pre-determined membrane staining intensity threshold. Decreasing the threshold on sum of percentages will include nuclei with partial membrane ring for assigning 2+ score to a tissue sample. It is necessary to reduce this threshold if the staining intensity is low or the device used to cut tissue sample is not sharp. Sectioning a biological sample with blunt devices might result in fragmented segments of membrane rings. Increasing the threshold on sum of percentages ensures that only nucleus with large segment of membrane ring is identified for scoring. In the case of over stained samples, it is required to identify complete ring stained cells to arrive at accurate medical diagnosis or life science and biotechnology experiment conclusion.

A membrane staining score 1+ is given if the sum of percentages of cells with complete stained membrane ring and partial stained membrane ring is more than pre-determined stained membrane segment threshold. Decreasing the stained membrane segment threshold will include nuclei with lesser segment of membrane ring for assigning 1+ score to a tissue sample. It is necessary to reduce this threshold if the staining intensity is low or the device used to cut tissue sample is not sharp. Sectioning a biological sample with blunt devices might result in fragmented segments of membrane rings. Increasing the stained membrane segment threshold ensures that only nuclei with greater segment of membrane staining are identified for1+ score. In the case of over stained samples, it is required to increase stained membrane segment threshold to arrive at accurate medical diagnosis or life science and biotechnology experiment conclusion.

A membrane staining score 0+ is given if the sum of percentages of cells with complete stained membrane ring and partial stained membrane ring is less than pre-determined stained membrane segment threshold.

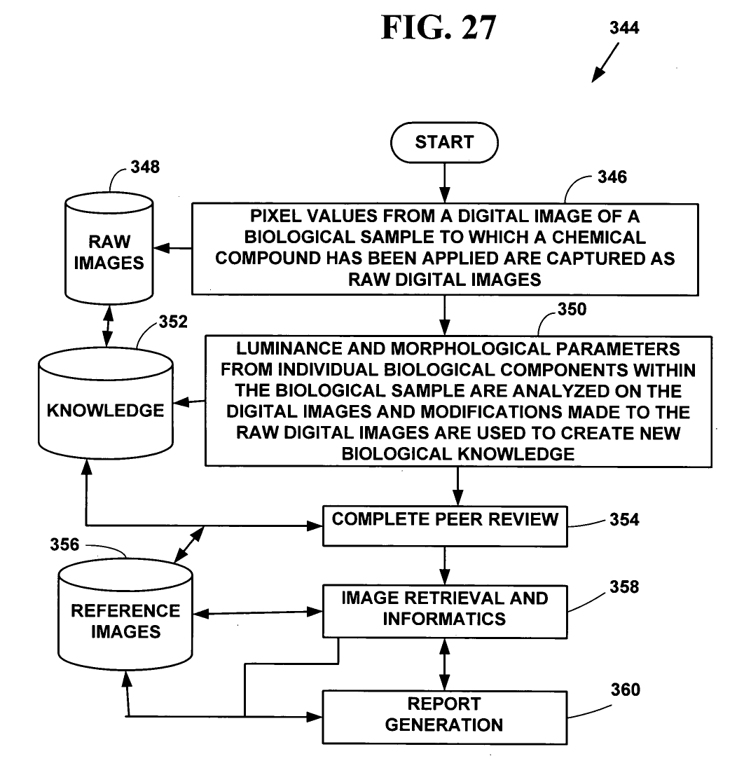

FIG. 27 is a block diagram illustrating an exemplary flow of data 344 in the automated biological sample analysis processing system 10. Pixel values from a digital image of a biological sample to which a chemical compound has been applied are captured 346 as raw digital images 348. The raw digital images are stored in raw image format in one or more image databases 18. Luminance and morphological parameters from individual biological components within the biological sample are analyzed on the digital image and modifications made to the raw digital images are used to create new biological knowledge 350 using the methods described herein. The new biological knowledge is stored in a knowledge database 352. Peer review of the digital image analysis and life science and biotechnology experiment results is completed 354. A reference digital image database 356 facilitates access of reference images from previous records of life science and biotechnology experiments at the time of peer review. Contents of the reference digital image database 356, information on the biological sample and analysis of current biological sample are available at an image retrieval and informatics module 358 that displays information on GUI 14. Conclusions of a medical diagnosis or prognosis or life science and biotechnology experiment are documented as one or more reports. Report generation 360 allows configurable fields and layout of the report. New medical knowledge is automatically created.

The present invention is implemented in software. The invention may be also be implemented in firmware, hardware, or a combination thereof, including software. However, there is no special hardware or software required to use the proposed invention. The methods and system described herein may provide the following: (1) Contrast and brightness parameters of luminance in a digital image are used instead of similar parameters from chrominance or color factors; (2) Segmenting color pixels based on a first peak in luminance histogram of enhanced image helps ensures that a large percentage of objects of clinical interest are separated; (3) Color correction is based on the color plane parameters (e.g., red color mean and blue color mean) of a background and helps ensure that background color seeped into clinical interest cells is reduced; (4) Computation of membrane pattern based on intensity of membrane pixels helps ensure that a robust and reliable membrane pattern recognition; and (5) Medical diagnostic or prognostic decisions are automatically formulated (e.g., HER-2/neu grades, etc.).

It should be understood that the programs, processes, methods and system described herein are not related or limited to any particular type of computer or network system (hardware or software), unless indicated otherwise. Various combinations of general purpose, specialized or equivalent computer components including hardware, software, and firmware and combinations thereof may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more fewer or equivalent elements may be used in the block diagrams.

The claims should not be read as limited to the described order or elements unless stated to that effect. In addition, use of the term “means” in any claim is intended to invoke 35 U.S.C. §112, paragraph 6, and any claim without the word “means” is not so intended.

Therefore, all embodiments that come within the scope and spirit of the following claims and equivalents thereto are claimed as the invention.

1. An automated method for biological sample analysis, comprising:
automatically analyzing luminance parameters from a digital image of a biological tissue sample to which a marker dye has been applied to determine one or more areas of interest in the biological tissue sample within the digital image;
automatically adjusting luminance parameters within the one or more determined areas of interest within the digital image to create one or more adjusted areas of interest;
automatically analyzing morphological parameters from individual biological components within the one or more adjusted areas of interest within the digital image; and
automatically formulating a medical diagnosis using the analyzed morphological parameters within the one or more adjusted areas of interest within the digital image.

2. The method of claim 1 further comprising a computer readable medium have stored therein instructions for causing a processor to execute the steps of the method.

3. The method of claim 1 wherein the marker dye includes immunohistochemical (IHC) staining.

4. The method of claim 1 wherein the biological tissue sample includes a plurality of human cells.

5. The method of claim 4 wherein the plurality of human cells includes one or more human cancer cells.

6. The method of claim 1 wherein the step of automatically analyzing luminance parameters includes analyzing a plurality of luminosities Y at a plurality of pixels within the digital image with:
Y=XG+YR+XB,
wherein X, Y and Z are pre-determined constants R, G and B are Red, Green and Blue color component values of a selected pixel within the digital image and X, Y and Z are predetermined constant values.

7. The method of claim 1 wherein the step of automatically analyzing luminance parameters includes determining an area of interest by excluding pixels which include biological tissue samples from background pixels in the digital image.

8. The method of claim 1 wherein the step of automatically adjusting luminance parameters adjusting luminance parameters within the one or more determined areas of interest includes:
modifying a contrast of the digital image based on statistics collected from the digital image to created a contrast modified digital image;
thresholding the contrast modified digital image to obtain a plurality of thresholded pixels including one or more areas of interest; and
correcting color components of the plurality of thresholded pixels within the one or more areas of interest in the contrast modified digital image.

9. The method of claim 8 wherein the step of modifying a contrast includes calculating for a plurality of pixels in the digital image:
R′=(R*(M+D)/K)+(X−M),
G′=(G*(M+D)/K)+(X−M),
B′=(B*(M+D)/K)+(X−M),
wherein K is a constant, R′, G′ and B′ are modified red (R), green (G) and blue (B) color components of a pixel, M is a mean and D is a standard deviation of a luminosity histogram calculated for the plurality of pixels in the digital image and X is a value of a pre-determined constant.

10. The method of claim 8 wherein the step of thresholding the contrast modified digital image includes:
calculating a luminosity histogram on the contrast modified digital image, wherein the luminosity histogram includes two or more peaks for grayscale values for a plurality of pixels in the digital image; and
selecting a plurality of pixels on the contrast modified digital image with a grayscale value less than or equal to a maximum grayscale value for a first peak in the luminosity histogram.

11. The method of claim 8 wherein the step of correcting color components includes:
calculating a correction factor C_{f},
wherein
C_{f}=(mean of a first color plane)/(mean of a second color plane) in the original digital image; and
correcting color components of the plurality of selected pixels within the one or more items of interest in the contrast modified digital image by multiplying the plurality of selected pixels by the correction factor C_{f}.

12. The method of claim 11 wherein the first color plane includes a red color plane and the second color plane include a blue color plane.

13. The method of claim 1 wherein step of automatically analyzing morphological parameters includes analyzing membranous rings around cell nuclei.

14. The method of claim 1 wherein the step of automatically formulating a medical diagnosis includes:
broadly classifying into two or more groups based on a presence of pre-determined pixels using the analyzed morphological parameters within the one or more adjusted areas of interest within the digital image;
detecting one or more predetermined analyzed morphological parameters within the one or more adjusted areas of interest within the digital image; and
preparing a final grading based on a completeness of the one or more predetermined analyzed morphological parameters.

15. The method of claim 14 wherein the broadly classifying step includes broadly classifying for human epidermal growth factor receptor HER-2/neu grading for a plurality of human tissue cells stained with Hematoxylin/Eosin (H/E) staining and grouping 0+ and 1+ HER-2/neu graded cells into a first group and 2+ and 3+ HER-2/neu graded cells into a second group based on a ratio of brown pixels versus blue pixels.

16. The method of claim 14 wherein the detecting step includes:
detecting a clear and complete cell membrane ring around a cell nucleus; and
detecting an intensity of a membranous pattern for the cell membrane ring.

17. The method of claim 1 wherein the step of automatically formulating a medical diagnosis includes formulating a medical diagnosis based on human epidermal growth factor receptor HER-2/neu grading for a plurality of human tissue cells stained with immunohistochemical (IHC) staining.

18. An automated method for biological sample analysis, comprising:
automatically segmenting morphological components from a biological tissue sample to which a marker dye has been applied within a digital image into one or more areas of interest;
automatically adjusting viewable characteristics of the segmented morphological components using one or more digital image processing techniques to create one or more adjusted areas of interest within the digital image; and
automatically computing a medical diagnosis grade based on the segmented morphological components within the one or more adjusted areas of interest within the digital image.

19. The method of claim 17 further comprising a computer readable medium having stored therein instructions for causing a processor to execute the steps of the method.

20. The method of claim 18 wherein the step of automatically segmenting morphological components includes segmenting cell nuclei and cell membranes from cytoplasm, fibrin and other components in the biological tissue sample within the digital image.

21. The method of claim 18 wherein the step of automatically adjusting viewable characteristics includes:
modifying a contrast of the digital image based on statistics collected from the digital image to created a contrast modified digital image;
thresholding the contrast modified digital image to obtain a plurality of thresholded pixels in the one or more areas of interest; and
correcting color components of the plurality of thresholded pixels within the one or more areas of interest in the contrast modified digital image to create one or more adjusted areas of interest within the digital image.

22. The method of claim 18 wherein the step of automatically computing a medical diagnosis grade includes computing a human epidermal growth factor receptor HER-2/neu grade based on continuity of cell membranous rings around cell nuclei within the one or more adjusted areas of interest within the digital image.

23. An automated method for biological sample analysis, comprising:
automatically analyzing luminance parameters from a digital image of a biological tissue sample to which a marker dye has been applied to determine one or more areas of interest in the biological tissue sample within the digital image;
automatically adjusting luminance parameters within the one or more determined areas of interest within the digital image to create one or more adjusted areas of interest;
automatically identifying a plurality of epithelial areas in the one or more adjusted areas of interest within the digital image for cell classification using cell membrane analysis;
automatically identifying a plurality of cell nuclei with the plurality epithelial areas in the one or more adjusted areas of interest within the digital image;
automatically identifying a plurality of cell membranes in the one or more adjusted areas of interest within the digital image;
automatically classifying the plurality of identified cell nuclei with a pre-determined classification scheme; and
automatically computing a medical diagnosis grade based on the classified cell nuclei.

24. The method of 23 further comprising a computer readable medium having stored therein instructions for causing a processor to execute the steps of the method.

25. The method of claim 23 wherein the step of automatically computing a medical diagnosis grade includes computing a human epidermal growth factor receptor HER-2/neu grade based on continuity of cell membranous rings around cell nuclei within the one or more adjusted areas of interest within the digital image.

26. The method of claim 25 wherein the HER-2/neu grade includes 0+, 1+, 2+ and 3+, HER-2/neu grading for a plurality of human tissue cells stained with IHC and counter stained with haematoxylin.

27. A biological sample analysis system, comprising in combination:
an automated analysis means for analyzing luminance parameters from a digital image of a biological tissue sample to which a marker dye has been applied to determine one or more areas of interest in the biological tissue sample within the digital image, for adjusting luminance parameters within the one or more determined areas of interest within the digital image to create one or more adjusted areas of interest, and for analyzing morphological parameters from individual biological components within the one or more adjusted areas of interest within the digital image; and
an automated medical diagnosis means for formulating a medical diagnosis using the analyzed morphological parameters within the one or more adjusted areas of interest within the digital image adjusted by the automated analysis means.

28. The biological sample analysis system of claim 27 wherein the automated medical diagnosis means formulates medical diagnosis based on human epidermal growth factor receptor HER-2/neu grading for a plurality of human tissue cells stained with immunohistochemical (IHC) staining and counterstained with haematoxylin using analyzed cell morphological parameters.

29. A biological sample analysis system, comprising in combination:
a module for automatically analyzing luminance parameters from a digital image of a biological tissue sample to which a marker dye has been applied to determine one or more areas of interest in the biological tissue sample within the digital image and for automatically adjusting luminance parameters within the one or more determined areas of interest within the digital image to create one or more adjusted areas of interest;
a module for automatically identifying a plurality of epithelial areas in the one or more adjusted areas of interest within the digital image for cell classification using cell membrane analysis, for automatically identifying a plurality of cell nuclei with the plurality epithelial areas in the one or more adjusted areas of interest within the digital image, for automatically identifying a plurality of cell membranes in the one or more adjusted areas of interest within the digital image, for automatically classifying the plurality of identified cell nuclei with a pre-determined classification scheme; and
a module for automatically computing a medical diagnosis grade based on the classified cell nuclei.

30. An automated method for biological sample analysis, comprising:
automatically analyzing luminance parameters from a digital image of a biological tissue sample to which a marker dye has been applied to determine one or more areas of interest in the biological tissue sample within the digital image;
automatically adjusting luminance parameters within the one or more determined areas of interest within the digital image to create one or more adjusted areas of interest;
automatically analyzing morphological parameters from individual biological components within the one or more adjusted areas of interest within the digital image; an
automatically formulating a medical diagnosis using the analyzed morphological parameters within the one or more adjusted areas of interest within the digital image and one or more diagnostic knowledge records from a knowledge database; and
automatically saving the formulated medical diagnosis in the knowledge database to create additional diagnostic knowledge.

31. The method of claim 1 further comprising a computer readable medium have stored therein instructions for causing a processor to execute the steps of the method.

32. An automated method for biological sample analysis, comprising:
automatically formulating a medical diagnosis using analyzed morphological parameters within one or more adjusted areas of interest within a digital image of a biological tissue sample to which a chemical compound has been applied;
providing the medical diagnosis to a medical professional for verification of the automatically formulated medical diagnosis; and
automatically saving the automatically formulated medical diagnosis in the knowledge database to create additional diagnostic knowledge.

33. The method of claim 32 further comprising a computer readable medium have stored therein instructions for causing a processor to execute the steps of the method.
